(19) United States
(12) Patent Application Publication
(10) Pub. No.: US 2010/0240630 A1
(43) Pub. Date: Sep. 23, 2010

(54) COMPOUNDS AND THERAPEUTIC USES THEREOF

(75) Inventors: Dange Vijay KUMAR, Salt Lake City, UT (US); Kraig M. Yager, Salt Lake City, UT (US); David A. Gerrish, Salt Lake City, UT (US); Christophe Hoarau, Salt Lake City, UT (US); Mark B. Anderson, Oakland, CA (US)

Correspondence Address: Myrexis, Inc.;c/o CPA Global, P.O. Box 52050, Minneapolis, MN, 55402, US

(73) Assignee: Myriad Pharmaceuticals, Inc.., Salt Lake City, UT (US)

(21) Appl. No.: 12/794,300

(22) Filed: Jun. 4, 2010

Related U.S. Application Data
(63) Continuation of application No. PCT/US2008/085592, filed on Dec. 4, 2008.
(60) Provisional application No. 61/005,274, filed on Dec. 4, 2007.
(60) Provisional application No. 61/079,950, filed on Jul. 11, 2008.

(51) Int. Cl. A61K 31/58; C07J 7/00; A61K 31/57; C07J 53/00; A61P 31/18; C07J 75/00; C12N 5/00

(52) U.S. Cl. 514/176; 552/511; 514/182; 540/47; 514/172; 435/375

(57) ABSTRACT

The invention relates to compounds, pharmaceutical compositions and methods useful for treating viral infection.

### CROSS-REFERENCE TO RELATED APPLICATIONS

This application is a continuation of International Application No. PCT/US2008/085592, filed Dec. 4, 2008 and published as WO 2009/073818, which claims the benefit of both U.S. Provisional Application Ser. No. 61/079,950, filed on Jul. 11, 2008 and U.S. Provisional Application Ser. No. 61/005,274, filed on Dec. 4, 2007; all of which are incorporated by reference herein in their entirety.

### FIELD OF THE INVENTION

The present invention relates generally to compounds, pharmaceutical compositions, and methods for treating (and delaying the onset of) diseases, particularly viral infection such as HIV infection and AIDS.

### BACKGROUND OF THE INVENTION

Viral infection of humans is a major health problem, and viral infection of domesticated animals is a major economic concern. Combating viral infection has proven to be highly effective in some cases but more difficult in others. Hepatitis B and C, human immunodeficiency virus (HIV), herpes simplex viruses, and influenza are just a few prominent members of a list of viruses that pose significant health threats worldwide. Treatments currently available for many viral infections are often associated with adverse side effects. In addition, antiviral therapeutics directed towards specific viral gene products frequently have the effect of driving the selection of viruses resistant to such therapeutics, and viral strains resistant to current methods of treatment are an increasing problem. Accordingly, there is a clear and ever-present need for new antiviral treatments.

A number of articles and patent publications disclose derivative compounds of betulinic acid that are useful for treating HIV infection, including U.S. Patent Publication No. 2006135495, PCT Publication No. WO/2008/057420, Huang et al., Antimicrobial Agents and Chemotherapy, 48:633-665 (2004), and Sun et al., J. Med. Chem., 45:4271-4275 (2002).

### BRIEF SUMMARY OF THE INVENTION

The present invention generally relates to compounds useful for treating viral infections, particularly HIV infection. Specifically, the present invention provides compounds of having Formula I

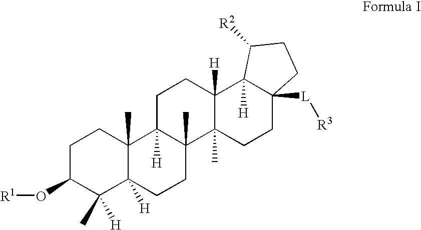

and pharmaceutically acceptable salts thereof,
wherein L, R¹, R², and R³ are as defined herein below.

The compounds of the present invention are effective HIV inhibitors, and are useful in inhibiting HIV infection and transmission. Thus, in a related aspect, the present invention also provides a method for treating viral infection, particularly HIV infection and AIDS, by administering to a patient in need of such treatment a therapeutically effective amount of a compound of the present invention.

Also provided is the use of a compound of Formula I for the manufacture of a medicament useful for therapy, particularly for treating HIV infection and AIDS. In addition, the present invention also provides a pharmaceutical composition having a compound of Formula I and one or more pharmaceutically acceptable excipients. A method for treating viral infection, particularly HIV infection and AIDS, by administering to a patient in need of the treatment the pharmaceutical composition is also encompassed.

In addition, the present invention further provides methods for inhibiting, or reducing the likelihood of, HIV transmission, or delaying the onset of the symptoms associated with HIV infection, or delaying the onset of AIDS, comprising administering an effective amount of a compound of the present invention, preferably in a pharmaceutical composition or medicament to an individual having an HIV infection, or at risk of HIV infection, or at risk of developing symptoms of HIV infection or AIDS.

The compounds of the present invention can be used in combination therapies. Thus, combination therapy methods are also provided for treating HIV infection, inhibiting, or reducing the likelihood of, HIV transmission, or delaying the onset of the symptoms associated with HIV infection, or delaying the onset of AIDS. Such methods comprise administering to a patient in need thereof a compound of the present invention, and together or separately, at least one other anti-HIV compound. For the convenience of combination therapy, the compound of the present invention can be administered together in the same formulation with such other anti-HIV compound. Thus, the present invention also provides a pharmaceutical composition or medicament for the combination therapy, comprising an effective amount of a first compound according to the present invention and an effective amount of at least one other anti-HIV compound, which is different from the first compound. Examples of antiviral compounds include, but are not limited to, protease inhibitors, reverse transcriptase inhibitors, integrase inhibitors, fusion inhibitors, immunomodulators, and vaccines.

In accordance with another aspect of the present invention, intermediate compounds of Formulae II, IIa and IIb are provided, which are useful in making the antiviral compounds of the present invention. Also provided are methods of synthesis of the antiviral compounds of the present invention including those using the intermediate compounds.

The foregoing and other advantages and features of the invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### DETAILED DESCRIPTION OF THE INVENTION

The present invention generally relates to compounds useful for treating viral infections, particularly HIV infection. Specifically, the present invention provides of compounds of having Formula I, and Formula Ia or Ib, which are useful in treating viral infections, particularly HIV infection. Also provided are compounds of Formulae II, IIa and IIb, which are useful in the manufacture of the compounds of Formula Ia. Thus, the compounds of the present invention are those having the following Formula I, Ia, Ib, II, IIa, or IIb:

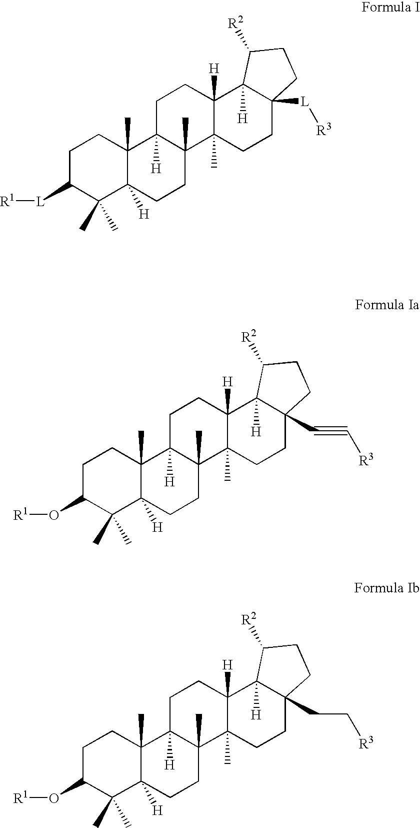

and pharmaceutically acceptable salts thereof, and

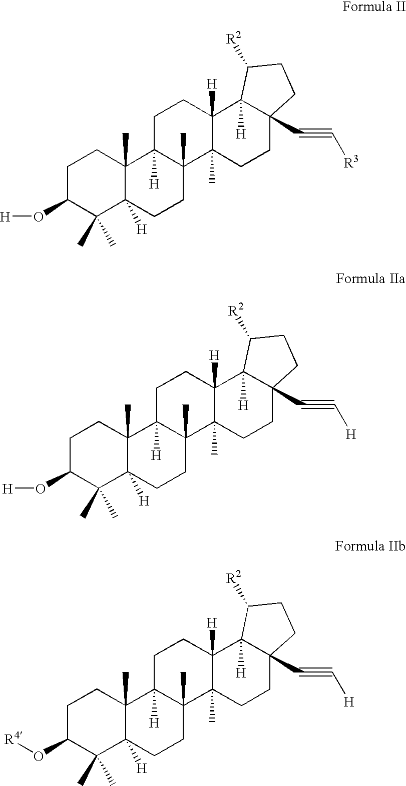

wherein:

L is ethylene or ethynylene;

R¹ is hydro, R¹¹—C(O)—, —S(O)R¹¹ or —S(O)OR¹¹, wherein R¹¹ is C₁₋₂₀ (preferably C₁₋₁₀, more preferably C₁₋₆) alkyl, C₂₋₂₀ (preferably C₂₋₁₀, more preferably C₂₋₆) alkenyl, or C₂₋₂₀ (preferably C₂₋₁₀, more preferably C₂₋₆) alkynyl, each being optionally substituted with one or more substituents independently chosen from the group of:

—C(O)R¹² where R¹² is —OH, C₁₋₆ alkoxy, C₁₋₆ alkenyloxy, C₁₋₆ alkynyloxy, C₃₋₆ cycloalkoxy;

carboxyalkoxy;

—C(O)—N(R¹³)(R¹⁴) where R¹³ and R¹⁴ are independently H, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is H, C₁₋₆ alkyl or aryl, or R¹³ and R¹⁴ together with the nitrogen atom they are linked to form a 3 to 6-membered heterocycle;

—N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;

—SO₃R¹⁵, where R¹⁵ is C₁₋₆ alkyl, aryl or heteroaryl; —NHSO₃R¹⁶, where R¹⁶ is C₁₋₆ alkyl, aryl, or heteroaryl; and —P(O)(OR¹⁷)₂ where R¹⁷ is H or C₁₋₆ alkyl,

wherein optionally two substituents (e.g., one alkyl and one hydroxyl) at one carbon atom of R¹¹ may, together with the one carbon atom they are attached to, form a 3 to 6-membered cycloalkyl. Preferably, R¹ is C₄₋₈ carboxyalkanoyl or C₄₋₈ carboxyalkenoyl, and more preferably is 3′,3′-dimethylsuccinyl or 3′,3′-dimethylglutaryl. Alternatively, R¹ can be R¹¹—X—C(O)— wherein X is O, NH or S, R¹¹ is C₁₋₂₀ (preferably C₁₋₁₀, more preferably C₁₋₆) alkyl, C₂₋₂₀ (preferably C₂₋₁₀, more preferably C₂₋₆) alkenyl, or C₂₋₂₀ (preferably C₂₋₁₀, more preferably C₂₋₆) alkynyl, each being optionally substituted with one or more substituents independently represented by —C(O)R¹² where R¹² is —OH, C₁₋₆ alkoxy, C₁₋₆ alkenyloxy, C₁₋₆ alkynyloxy, C₃₋₆ cycloalkoxy or heterocycle.

R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopropyl, 1′,2′-dihydroxyisopropyl, and 1′-pyrrolidinyl-2′-hydroxyisopropyl;

R³ is hydro or an optionally substituted aryl, heteroaryl, carbocycle or heterocycle. For example, R³ can be an aryl, heteroaryl, carbocycle or heterocycle substituted with one or more (e.g., 1, 2, 3, 4 or 5) substituents independently chosen from the group of hydroxyl, mercapto, halo (F, Cl, Br, or I), cyano, nitro, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ alkylthio, C₁₋₆ hydroxyalkyl, C₁₋₆ thioalkyl, alkoxyalkyl, carboxylic acid, carboxylic acid bioisosteres, carboxyalkyl, carboxyalkoxy, carboxyalkenyl, carboxyalkynyl, alkanoyl, alkylthiocarnonyl, carboxylalkoxyalkanoyl, C-carboxy, O-carboxy, O-carbamyl, N-carbamyl, O-thiocarbamyl, N-thiocarbamyl, amino, aminoalkyl, alkylamino, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH); Preferably when L is ethylene, R³ is an optionally substituted aryl, preferably an aryl having at least one substituent that is carboxylic acid.

R⁴′ is an ester of R⁴ that is C4₋₈ carboxyalkanoyl, C₄₋₈ carboxyalkenoyl or C₄₋₈ carboxyalkoxyalkanoyl.

In some embodiments, L is ethynylene (see Formula Ia), and in some other embodiments L is ethylene (see Formula Ib).

In some embodiments, R¹ is C₄₋₈ carboxyalkanoyl, C₄₋₈ carboxyalkenoyl, or C₄₋₈ carboxyalkoxyalkanoyl.

In some embodiments, R¹ and R⁴ are chosen from the group consisting of:

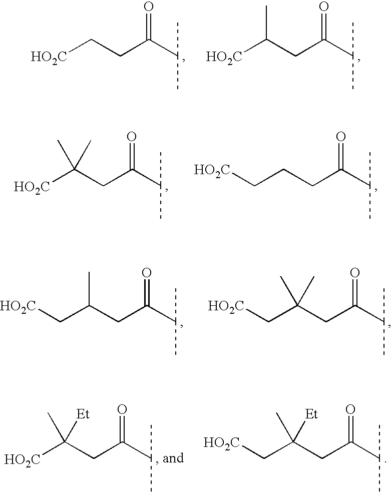

In some embodiments, R¹ and R⁴ are —C(═O)—(CH₂)ₘ—C(CH₃)₂—COOH or —C(═O)—(CH₂)ₘ—C(CH₃)₂—(CH₂)ₙ COOH, wherein m and n are independently an integer from 0-10, preferably 0, 1 or 2. In some embodiments, R¹ and R⁴ are 3′,3′-dimethylsuccinyl or 3′,3′-dimethylglutaryl.

In some embodiments, R² is isopropenyl, isopropyl, 1′-hydroxyisopropyl, 2′-hydroxyisopryl, 1′,2′-dihydroxyisopropyl, or 1′-pyrrolidinyl-2′-hydroxyisopropyl. In some embodiments, R² is —C(═CH₂)—CH₃ or —CH(CH₃)₂.

In some embodiments, R³ is an aryl, heteroaryl, carbocycle or heterocycle having a substituent at an ortho position relative to (or adjacent to) the position where L (ethynylene or ethylene) is attached to.

In some embodiments, R³ is an aryl, heteroaryl, carbocycle or heterocycle substituted with one or more (1, 2, 3, 4, or 5) substituents independently chosen from the group consisting of hydroxyl, halo (F, Cl, Br, I), C₁₋₆ alkyl, C₁₋₆ alkoxy, C₁₋₆ alkylthio, carboxylic acid, C-carboxy, C-amido, aminosulfonyl, sulfonyl, and —COOH bioisosteres, preferably at least one substituent being at an ortho position relative to (or adjacent to) the position where L (ethynylene or ethylene) is attached to.

In some embodiments, R³ is phenyl, thiophenyl, furanyl, benzofuranyl, benzothiophenyl, isoxazolyl, pyridinyl, pyrazinyl, pyrimidinyl,

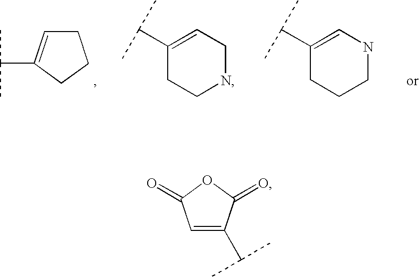

optionally substituted with one or more (1, 2, 3, 4, or 5) substituents independently chosen from the group consisting of hydroxyl, halo (F, Cl, Br, I), C₁₋₆ alkyl, C₁₋₆ alkoxy, C₁₋₆ alkylthio, carboxylic acid, C-carboxy, C-amido, aminosulfonyl, sulfonyl, and —COOH bioisosteres, preferably at least one substituent being at an ortho position relative to (or adjacent to) the position where L (ethynylene or ethylene) is attached to.

In some embodiments, R³ is phenyl,

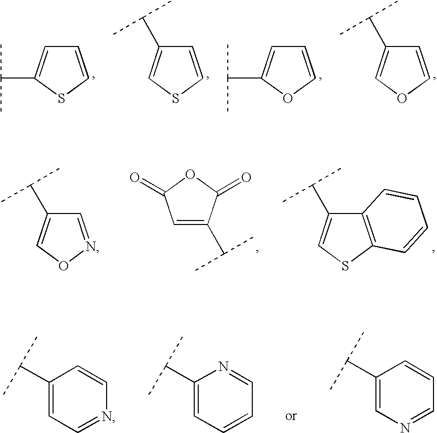

optionally substituted with one or more (1, 2, 3, 4, or 5) substituents independently chosen from the group consisting of hydroxyl, halo (F, Cl, Br, I), C₁₋₆ alkyl, C₁₋₆ alkoxy, C₁₋₆ alkylthio, carboxylic acid, C-carboxy, C-amido, aminosulfonyl, sulfonyl, and carboxylic acid bioisosteres, preferably at least one substituent being at an ortho position relative to (or adjacent to) the position where L (ethynylene or ethylene) is attached to.

In some embodiments, R³ is an aryl, heteroaryl, carbocycle or heterocycle (e.g., phenyl, thiophenyl, furanyl, pyridinyl, benzothiophenyl,

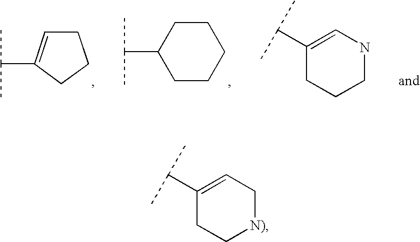

substituted with a substitutent at an ortho position relative to the position where L (ethynylene or ethylene) is attached to, chosen from hydroxyl, mercapto, C₁₋₆ alkoxy, C₁₋₆ haloalkoxy, C₁₋₆ alkylthio, carboxylic acid, carboxylic acid bioisosteres, carboxyalkyl, carboxyalkenyl, carboxyalkynyl, carboxyalkoxy, alkanoyl, alkylthiocarnonyl, carboxylalkoxyalkanoyl, C-carboxy, O-carboxy, O-carbamyl, N-carbamyl, O-thiocarbamyl, N-thiocarbamyl, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH).

In some embodiments, R³ is an aryl, heteroaryl, carbocycle or heterocycle (e.g., phenyl, thiophenyl, furanyl, pyridinyl, benzothiophenyl,

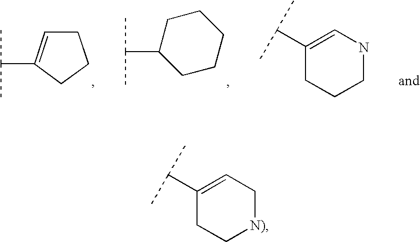

substituted with a substitutent that is carboxylic acid or a carboxylic acid bioisostere at an ortho position relative to the position where L (ethynylene or ethylene) is attached to.

In some embodiments, R³ is an aryl, heteroaryl, carbocycle or heterocycle (e.g., phenyl, thiophenyl, furanyl, pyridinyl, benzothiophenyl,

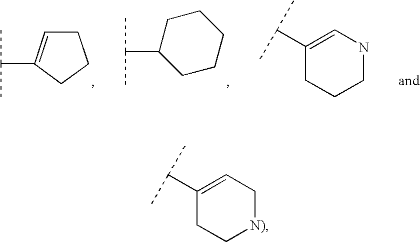

substituted with (1) a first substitutent at an ortho position relative to the position where L (ethynylene or ethylene) is attached to, wherein the first substituent is chosen from hydroxyl, mercapto, C₁₋₆ alkoxy, C₁₋₆ haloalkoxy, C₁₋₆ alkylthio, carboxylic acid, carboxylic acid bioisosteres, carboxyalkyl, carboxyalkenyl, carboxyalkynyl, carboxyalkoxy, alkanoyl, alkylthiocarnonyl, carboxylalkoxyalkanoyl, C-carboxy, O-carboxy, O-carbamyl, N-carbamyl, O-thiocarbamyl, N-thiocarbamyl, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH), and optionally (2) one or more (1, 2, 3 or 4) additional substituents independently chosen from the group of hydroxyl, mercapto, halo (F, Cl, Br, or I), cyano, nitro, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ alkylthio, C₁₋₆ hydroxyalkyl, C₁₋₆ thioalkyl, alkoxyalkyl, carboxylic acid, carboxylic acid bioisosteres, carboxyalkyl, carboxyalkoxy, carboxyalkenyl, carboxyalkynyl, alkanoyl, alkylthiocarnonyl, carboxylalkoxyalkanoyl, C-carboxy, O-carboxy, O-carbamyl, N-carbamyl, O-thiocarbamyl, N-thiocarbamyl, amino, aminoalkyl, alkylamino, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH).

In some embodiments, R³ is an aryl, heteroaryl, carbocycle or heterocycle (e.g., phenyl, thiophenyl, furanyl, pyridinyl, benzothiophenyl,

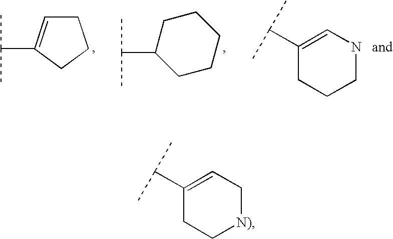

substituted with (1) a first substitutent that is carboxylic acid or a carboxylic acid bioisostere at an ortho position relative to the position where L (ethynylene or ethylene) is attached to, and optionally (2) one or more (1, 2, 3 or 4) additional substituents independently chosen from the group of hydroxyl, mercapto, halo (F, Cl, Br, or I), cyano, nitro, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ alkylthio, C₁₋₆ hydroxyalkyl, C₁₋₆ thioalkyl, alkoxyalkyl, carboxylic acid, carboxylic acid bioisosteres, carboxyalkyl, carboxyalkoxy, carboxyalkenyl, carboxyalkynyl, alkanoyl, alkylthiocarnonyl, carboxylalkoxyalkanoyl, C-carboxy, O-carboxy, O-carbamyl, N-carbamyl, O-thiocarbamyl, N-thiocarbamyl, amino, aminoalkyl, alkylamino, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH).

Specific examples of R³ are provided in Table 1 below:

TABLE 1
Examples of R³

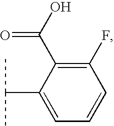

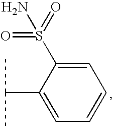

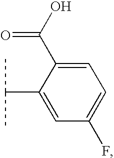

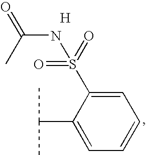

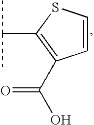

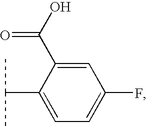

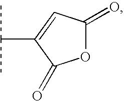

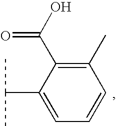

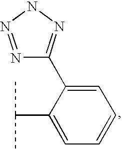

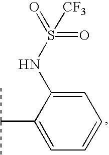

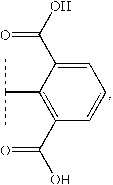

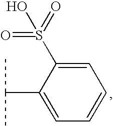

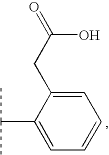

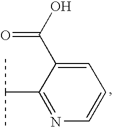

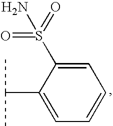

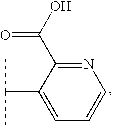

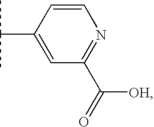

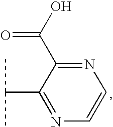

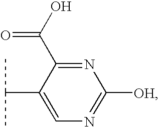

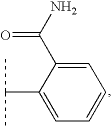

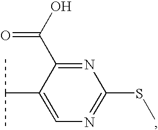

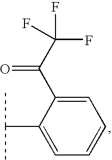

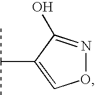

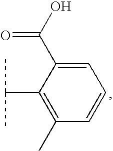

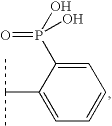

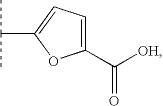

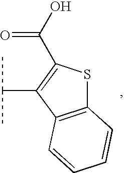

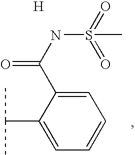

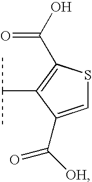

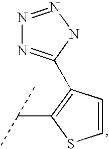

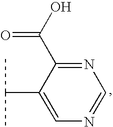

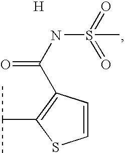

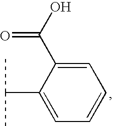

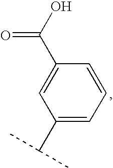

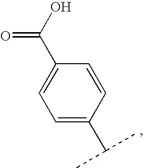

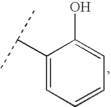

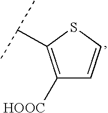

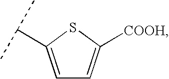

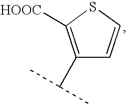

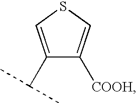

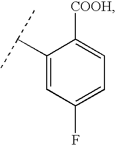

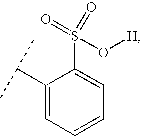

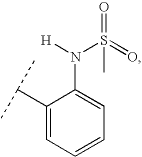

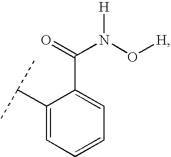

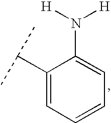

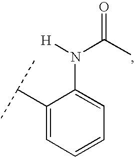

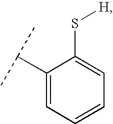

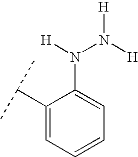

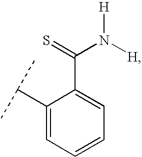

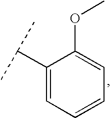

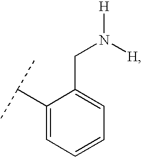

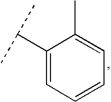

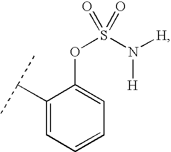

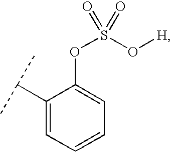

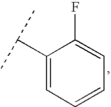

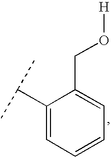

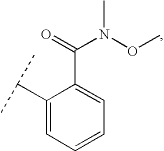

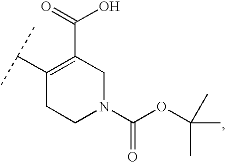

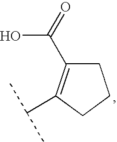

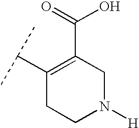

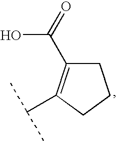

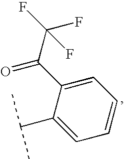

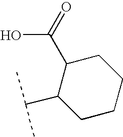

In preferred embodiments, compounds are provided according to any of the above formulae and having an IC₅₀ of less than about 10 μM, 5 μM, 2,500 nM, 500 nM, 300 nM, 200 nM, preferably less than about 100 nM, and most preferably less than about 80 nM, as determined in the MT4 assay in Example 2.

A pharmaceutically acceptable salt of the compound of the present invention is exemplified by a salt with an inorganic acid and/or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

Additionally, the compounds of the present invention can contain asymmetric carbon atoms and can therefore exist in racemic and optically active forms. Thus, optical isomers or enantiomers, racemates, and diastereomers are also encompassed, so long as the stereochemistry of the core structure of the compounds is equivalent to that of betulin. The methods of present invention include the use of all such isomers and mixtures thereof. The present invention encompasses any isolated racemic or optically active form of compounds described above, or any mixture thereof, which possesses anti-viral activity.

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

The term “bioisostere”, as used herein, generally refers to compounds or moieties that have chemical and physical properties producing broadly similar biological properties. Examples of carboxylic acid bioisosteres include, but are not limited to, carboxyalkyl, carboxylic acid ester, tetrazole, oxadiazole, isoxazole, hydroxythiadiazole, thiazolidinedione, oxazolidinedione, sulfonamide, aminosulfonyl, sulfonamidecarbonyl, C-amido, sulfonylcarboxamide, phosphonic acid, phosphonamide, phosphinic acid, sulfonic acid, alkanoylaminosulfonyl, mercaptoazole, trifluoromethylcarbonyl, and cyanamide.

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). An alkyl group may be in unsubstituted form or substituted form with one or more substituents (generally one to three substitutents except in the case of halogen substituents, e.g., perchloro). For example, a C₁₋₆ alkyl group refers to a straight or branched aliphatic group containing 1 to 6 carbon atoms (e.g., include methyl, ethyl, propyl, isopropyl, butyl, sec-butyl, tent-butyl, 3-pentyl, and hexyl), which may be optionally substituted. The term “alkylene” as used herein means a saturated aliphatic hydrocarbon straight chain or branched chain group having 1 to 20 carbon atoms having two connecting points. For example, “ethylene” represents the group —CH₂CH₂— or —(CH₃)CH—.

The term “alkenyl” as employed herein by itself or as part of another group means a straight or branched chain radical of 2-10 carbon atoms, unless the chain length is limited thereto, including at least one double bond between two of the carbon atoms in the chain. The alkenyl group may be in unsubstituted form or substituted form with one or more substituents (generally one to three substitutents except in the case of halogen substituents, e.g., perchloro or perfluoroalkyls). For example, a C₁₋₆ alkenyl group refers to a straight or branched chain radical containing 1 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which may be optionally substituted). The term “alkenylene” as used herein means an alkenyl group having two connecting points. For example, “ethenylene” represents the group —CH═CH— or —(CH═)C—.

The term “alkynyl” as used herein by itself or as part of another group means a straight or branched chain radical of 2-10 carbon atoms, unless the chain length is limited thereto, wherein there is at least one triple bond between two of the carbon atoms in the chain. The alkynyl group may be in unsubstituted form or substituted form with one or more substituents (generally one to three substitutents except in the case of halogen substituents, e.g., perchloro or perfluoroalkyls). For example, a C₁₋₆ alkynyl group refers to a straight or branched chain radical containing 1 to 6 carbon atoms and having at least one triple bond between two of the carbon atoms in the chain (e.g., ethynyl, 1-propynyl, 1-methyl-2-propynyl, 2-propynyl, 1-butynyl and 2-butynyl, which may be optionally substituted). The term “alkynylene” as used herein means an alkynyl having two connecting points. For example, “ethynylene” represents the group —CH≡CH—.

The term “carbocycle” as used herein by itself or as part of another group means cycloalkyl and non-aromatic partially saturated carbocyclic groups such as cycloalkenyl and cycloalkynyl. A carbocycle may be in unsubstituted form or substituted form with one or more substituents so long as the resulting compound is sufficiently stable and suitable for the treatment method of the present invention.

The term “cycloalkyl” as used herein by itself or as part of another group refers to a fully saturated 3- to 8-membered cyclic hydrocarbon ring (i.e., a cyclic form of an unsubstituted alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, or a spiral ring. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl group may be unsubstituted or substituted with one or more substitutents so long as the resulting compound is sufficiently stable and suitable for the treatment method of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring having a double bond therein (i.e., a cyclic form of an unsubstituted alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group may be in unsubstituted form or substituted form with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of carbon atoms with such other rings) (“polycyclic heterocylce”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be in unsubstituted form or substituted form with one or more substituents so long as the resulting compound is sufficiently stable and suitable for the treatment method of the present invention. Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be in unsubstituted form or substituted form with one or more substituents so long as the resulting compound is sufficiently stable and suitable for the treatment method of the present invention.

The term “heteroaryl” as employed herein refers to a stable aromatic ring having up to 7 ring atoms with 1, 2, 3 or 4 hetero ring atoms in the ring which are oxygen, nitrogen or sulfur or a combination thereof (“monocylic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be in unsubstituted form or substituted form with one or more substituents so long as the resulting compound is sufficiently stable and suitable for the treatment method of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-c]pyrimidin-4-one, pyrazolo[1,5-c]pyrimidinyl, including without limitation pyrazolo[1,5-c]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, and iodo.

As used herein, the term “hydro” refers to a hydrogen atom (—H group).

As used herein, the term “hydroxyl” refers to an —OH group.

As used herein, the term “alkoxy” refers to an —O—C₁₋₁₂ alkyl. Lower alkoxy refers to —O-lower alkyl groups.

As used herein, the term “cycloalkyloxy” refers to an —O-cycloakyl group.

As used herein, the term “aryloxy” refers to both an —O-aryl group.

The term “heteroaryloxy” refers to an —O-heteroaryl group.

The terms “arylalkoxy” and “heteroarylalkoxy” are used herein to mean an alkoxy group substituted with an aryl group and a heteroaryl group, respectively.

As used herein, the term “mercapto” group refers to an —SH group.

The term “alkylthio” group refers to an —S-alkyl group.

The term “arylthio” group refers to an —S-aryl group.

The term “arylalkyl” is used herein to mean an alkyl group substituted with an aryl group. Examples of arylalkyl include benzyl, phenethyl or naphthylmethyl.

The term “heteroarylalkyl” is used herein to mean an alkyl group substituted with a heteroaryl group.

The term “arylalkenyl” is used herein to mean an alkenyl group substituted with an group.

“Heteroarylalkenyl” means an alkenyl group substituted with a heteroaryl group.

“Arylalkynyl” means an alkynyl having a substituent that is an aryl group.

The term “heteroarylalkynyl” is used herein to mean an alkynyl group substituted with a heteroaryl group.

“Haloalkyl” means an alkyl group that is substituted with one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

As used herein, the term “carbonyl” group refers to a —C(═O)— group.

The term “thiocarbonyl” group refers to a —C(═S)— group.

“Alkanoyl” refers to an alkyl-C(═O)— group.

The term “acetyl” group refers to a —C(═O)CH₃ group.

“Alkylthiocarnonyl” refers to an alkyl-C(═S)— group.

The term “cycloketone” refer to a carbocycle or heterocycle group in which one of the carbon atoms which form the ring has a “═O” bonded to it; i.e. one of the ring carbon atoms is a —C(═O) group.

The term “O-carboxy” group refers to a R″C(═O)O— group, where R″ is as defined herein below.

The term “C-carboxy” group refers to a —C(═O)OR″ groups where R″ is as defined herein below.

The term “carboxylic acid” refers to —COOH.

The term “ester” is a C-carboxy group, as defined herein, wherein R″ is any of the listed groups other than hydro.

The term “C-carboxy salt” refers to a —C(═O)O⁻M⁺ group wherein M⁺ is selected from the group consisting of lithium, sodium, magnesium, calcium, potassium, barium, iron, zinc, copper, and ammonium.

The term “carboxyalkyl” refers to —C₁₋₆ alkylene-C(═O)OR″ (that is, a C₁₋₆ alkyl group connected to the main structure wherein the alkyl group is substituted with —C(═O)OR″ with R″ being defined herein below). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein below.

The term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

The term “carboxyalkoxy” refers to —O—(CH₂)ₙC(═O)OR″ wherein r is 1-6, and R″ is as defined herein below.

“Cₓ carboxyalkanoyl” means a carbonyl group (—(O═)C—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—(O═)C—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein below.

“Amino” refers to an —NR^{x}R^{y} group, with R^{x} and R^{y} as defined herein.

“Alkylamino” means an amino group with a substituent being a C₁₋₆ alkyl.

“Aminoalkyl” means an alkyl group connected to the main structure of a molecule where the alkyl group has a substituent being amino.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

The term “O-carbamyl” refers to a —OC(═O)N(R^{x})(R^{y}) group with R^{x} and R^{y} as defined herein.

The term “N-carbamyl” refers to a R^{y} OC(═O)N(R^{x})— group, with R^{x} and R^{y} as defined herein.

The term “O-thiocarbamyl” refers to a —OC(═S)N(R^{x})(R^{y}) group with R^{x} and R^{y} as defined herein.

The term “N-thiocarbamyl” refers to a R^{x}OC(═S)NR^{y}— group, with R^{x} and R^{y} as defined herein.

“C-amido” refers to a —C(═O)N(R^{x})(R^{y}) group with R^{x} and R^{y} as defined herein.

“N-amido” refers to a R^{x}C(═O)N(R^{y})— group with R^{x} and R^{y} as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The term “cyano” refers to a —C≡N group.

The term “cyanato” refers to a —CNO group.

The term “isocyanato” refers to a —NCO group.

The term “thiocyanato” refers to a —CNS group.

The term “isothiocyanato” refers to a —NCS group.

The term “sulfinyl” refers to a —S(═O)R″ group, where R″ is as defined herein below.

The term “sulfonyl” refers to a —S(═O)₂R″ group, where R″ is as defined herein below.

The term “sulfonamide” refers to a —(R^{x})N—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{Y})N—S(═O)₂—O— group with R^{x} and R^{y} as defined herein.

“Sulfonamidecarbonyl” means R″—S(═O)₂—N(R^{x})—C(═O)— with R″ and R^{x} as defined herein below.

“Alkanoylaminosulfonyl” refers to an alkyl-C(═O)—N(R^{x})—S(═O)₂— group with R^{x} as defined herein below.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is selected from the group consisting of hydro, alkyl, cycloalkyl, aryl, heteroaryl and heterocycle, each being optionally substituted.

R^{x}, R^{y}, and R^{z} are independently selected from the group consisting of hydro and optionally substituted alkyl.

The term “methylenedioxy” refers to a —OCH2O— group wherein the oxygen atoms are bonded to adjacent ring carbon atoms.

The term “ethylenedioxy” refers to a —OCH2CH2O— group wherein the oxygen atoms are bonded to adjacent ring carbon atoms.

The present invention provides methods for treating viral infection, particularly HIV infection, delaying the onset of HIV infection, treating AIDS, delay the onset of AIDS, by treating a patient (either a human or another animal) in need of the treatment, with a compound of the present invention.

As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blood animal, particularly mammal, more particularly a human a pharmaceutical composition comprising an effective amount of a compound according to the present invention.

As used herein, the term “HIV infection” generally encompasses infection of a host animal, particularly a human host, by the human immunodeficiency virus (HIV) family of retroviruses including, but not limited to, HIV-1, HIV-2, HIV I (also known as HTLV-III), HIV II (also known as LAV-1), HIV III (also known as LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or delay the onset of AIDS or AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4 T-cells), and AIDS (which is defined by more serious AIDS-defining illnesses and/or a decline in the circulating CD4 T-cell count to below a level that is compatible with effective immune function).

The term “delaying the onset of HIV infection” means treating an individual who (1) is at risk of infection by HIV, or (2) is suspected of infection by HIV or of exposure to HIV, or (3) has suspected past exposure to HIV, to delay the onset of acute primary infection syndrome by at least three months. As is known in the art, clinical findings typically associated with acute primary infection syndrome may include an influenza-like illness with fevers, malaise, nausea/vomiting/diarrhea, pharyngitis, lymphadenopathy, myalgias, and neurologic symptoms such as headache, encephalitis, etc. The individuals at risk may be people who perform any of following acts: contact with HIV-contaminated blood, blood transfusion, exchange of body fluids, “unsafe” sex with an infected person, accidental needle stick, injection of drug with contaminated needles or syringes, receiving a tattoo or acupuncture with contaminated instruments, or transmission of the virus from a mother to a baby during pregnancy, delivery or shortly thereafter. The term “delaying the onset of HIV infection” also encompasses treating a person who has not been diagnosed as having HIV infection but is believed to be at risk of infection by HIV, or has been exposed to HIV through contaminated blood, etc.

In addition, the term “delay the onset of AIDS” means delaying the onset of AIDS (which is characterized by more serious AIDS-defining illnesses and/or a decline in the circulating CD4 cell count to below a level that is compatible with effective immune function, i.e. below about 200/μl) and/or AIDS-related conditions, by treating an individual (1) at risk of infection by HIV, or suspected of being infected with HIV, or (2) having HIV infection but not AIDS, to delay the onset of AIDS by at least six months. Individuals at risk of HIV infection may be those who are suspected of past exposure, or considered to be at risk of present or future exposure, to HIV by, e.g., contact with HIV-contaminated blood, blood transfusion, transplantation, exchange of body fluids, “unsafe” sex with an infected person, accidental needle stick, receiving a tattoo or acupuncture with contaminated instruments, or transmission of the virus from a mother to a baby during pregnancy, delivery or shortly thereafter.

The term “treating AIDS” means treating a patient who exhibits more serious AIDS-defining illnesses and/or a decline in the circulating CD4 cell count to below a level that is compatible with effective immune function (typically below about 200/μl). The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

For example, a carrier of HIV can be identified by conventional diagnostic techniques known in the art, and the identified carrier can be treated with a compound of the present invention, preferably in a pharmaceutical composition having a pharmaceutically acceptable carrier.

In one aspect, the present invention provides methods for combination therapy for treating viral infection, particularly HIV infection, delaying the onset of HIV infection, treating AIDS, delay the onset of AIDS, by treating a patient (either a human or another animal) in need of the treatment, with a compound of the present invention together with one or more other anti-HIV agents. Such other anti-HIV agents include those agents targeting a viral protein such as viral protease, reverse transcriptase, integrase, envelope protein (e.g., gp120 and gp41 for anti-fusion or homolog thereof), or a host cell protein such as CCR5, CXCR4, etc. Thus, examples of such other antiviral compounds include, but are not limited to, protease inhibitors, nucleoside reverse transcriptase inhibitors, non-nucleoside reverse transcriptase inhibitors, integrase inhibitors, fusion inhibitors, and a combination thereof. In the combination therapy, the compound of the present invention can be administered separately from, or together with the one or more other anti-HIV agents.

In another aspect, the present invention further provides a medicament or a pharmaceutical composition having a therapeutically or prophylactically effective amount of a compound or a pharmaceutically acceptable salt thereof according to the present invention.

Typically, compounds according to the present invention can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be determined based on the effective daily amount and the pharmacokinetics of the compounds. In the case of combination therapy, a therapeutically effective amount of one or more other antiviral compounds can be administered in a separate pharmaceutical composition, or alternatively included in the pharmaceutical composition according to the present invention which contains a compound according to the present invention. The pharmacology and toxicology of many of such other antiviral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage range set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

For oral delivery, the active compounds can be incorporated into a formulation that includes pharmaceutically acceptable carriers such as binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. See, e.g., Phillips et al., J. Pharmaceut. Sci., 73:1718-1720 (1984).

The active compounds can also be incorporated into a prodrug, e.g., conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Another typical prodrug form is an ester of the parent compound, as is generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated, so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally speaking, the compounds of the present invention can be synthesized using methods known in the art combined with the disclosure herein. In general, many compounds of the invention can be synthesized according to Scheme 1 or Scheme 2 below, wherein R¹, R², and R³ are as defined above in connection with Formulae I, Ia, Ib, II, IIa, and IIb, including the specific embodiments thereof, wherein if R³ has any carboxylic acid moiety R^{3′} is an ester of R³, and if R³ does not have any carboxylic acid group, R^{3′} is same as R³, and wherein R⁴ is C₄₋₈ carboxyalkanoyl, C₄₋₈ carboxyalkenoyl, or C₄₋₈ carboxyalkoxyalkanoyl, and, and R^{4′} is an ester of R⁴. R¹ can be same as R⁴, and preferably R¹ and R⁴ are chosen from the group consisting of:

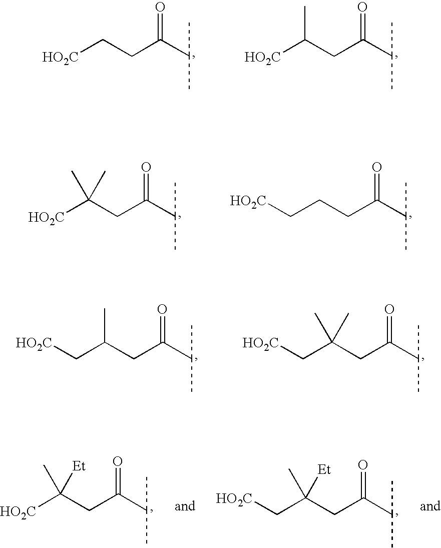

more preferably R¹ and R⁴ are —C(═O)—(CH₂)ₘ—C(CH₃)₂—COOH or —C(═O)—(CH₂)ₘ—C(CH₃)₂—(CH₂)ₙ—COOH, wherein m and n are independently an integer from 0-10 (e.g., 0, 1 or 2), and most preferably R¹ and R⁴ are 3′,3′-dimethylsuccinyl or 3′,3′-dimethylglutaryl.

Specifically, in Scheme 1,

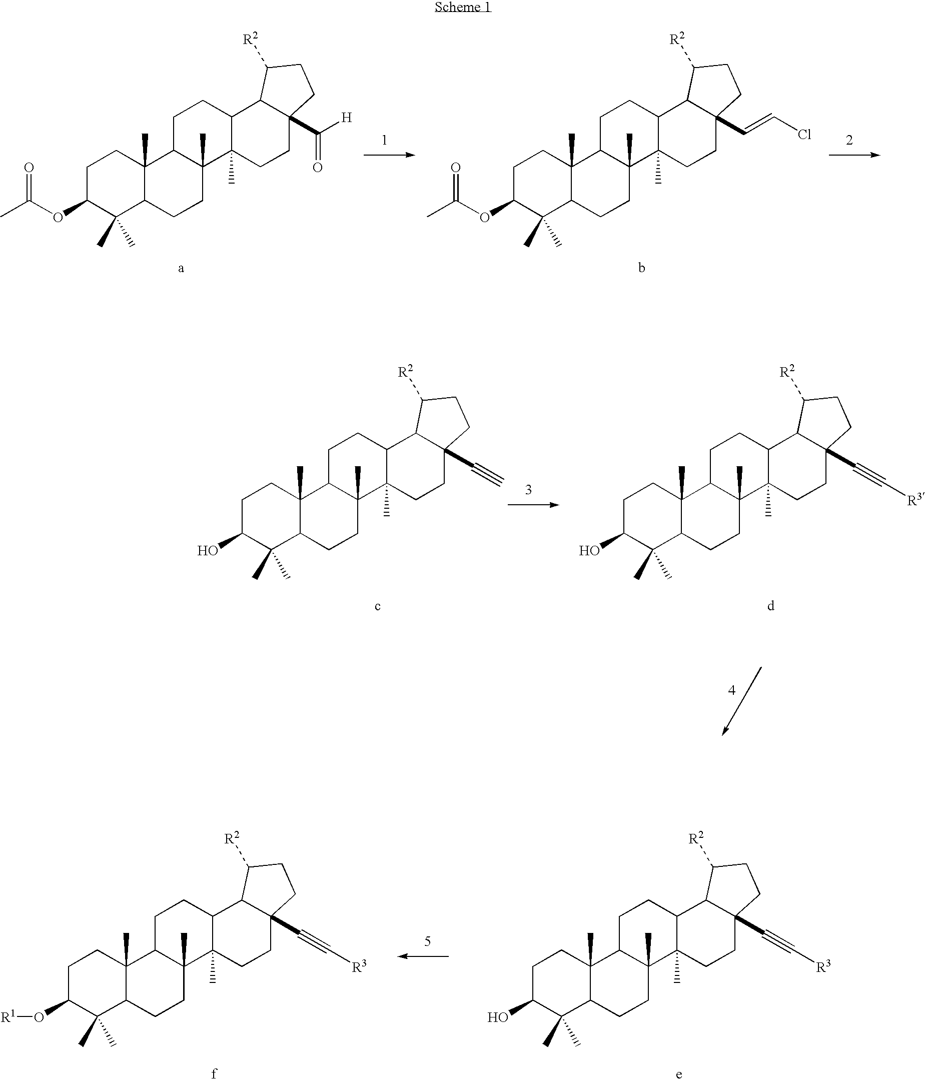

the method may start with a commercially available aldehyde compound a, wherein R² is isopropyl or isopropenyl. Alternatively, compound a can be prepared by (1) adding a protecting group to the C-3 position of betulin, and (2) then converting the —CH₂OH group at C-28 position to —CHO. The —CHO group at C-28 of compound a is then converted into a vinyl chloride moiety by Wittig olefination reaction (Compound b) (step 1). In step 2, Compound b is then treated with an alkyl lithium in a solvent producing Compound c having an alkynyl moiety at the C-28 position and a hydroxyl group at the C-3 position. In step 3, a R^{3′} moiety is attached to the alkynyl group of Compound c forming Compound d. This can be done, e.g., by reacting Compound c with an aryl halide or heteroaryl halide or carbocyclyl halide or heterocyclyl halide in a transition metal-catalyzed reaction. In step 4, the R^{3′} group in Compound d is converted into the R³ moiety by, e.g., removing any protection group in R^{3′} thus producing Compound e. This can be accomplished, e.g., by the hydrolysis of the protection group (e.g., an ester) by saponification in a basic condition. Finally, in step 5, Compound e is converted at C-3 position to R¹—O— producing Compound f. This is can be accomplished by, e.g., reacting Compound e with an appropriate compound having a carboxylic acid group, e.g., 2,2-dimethylsuccinic anhydride.

Alternatively, compounds can be synthesized using Scheme 2 below.

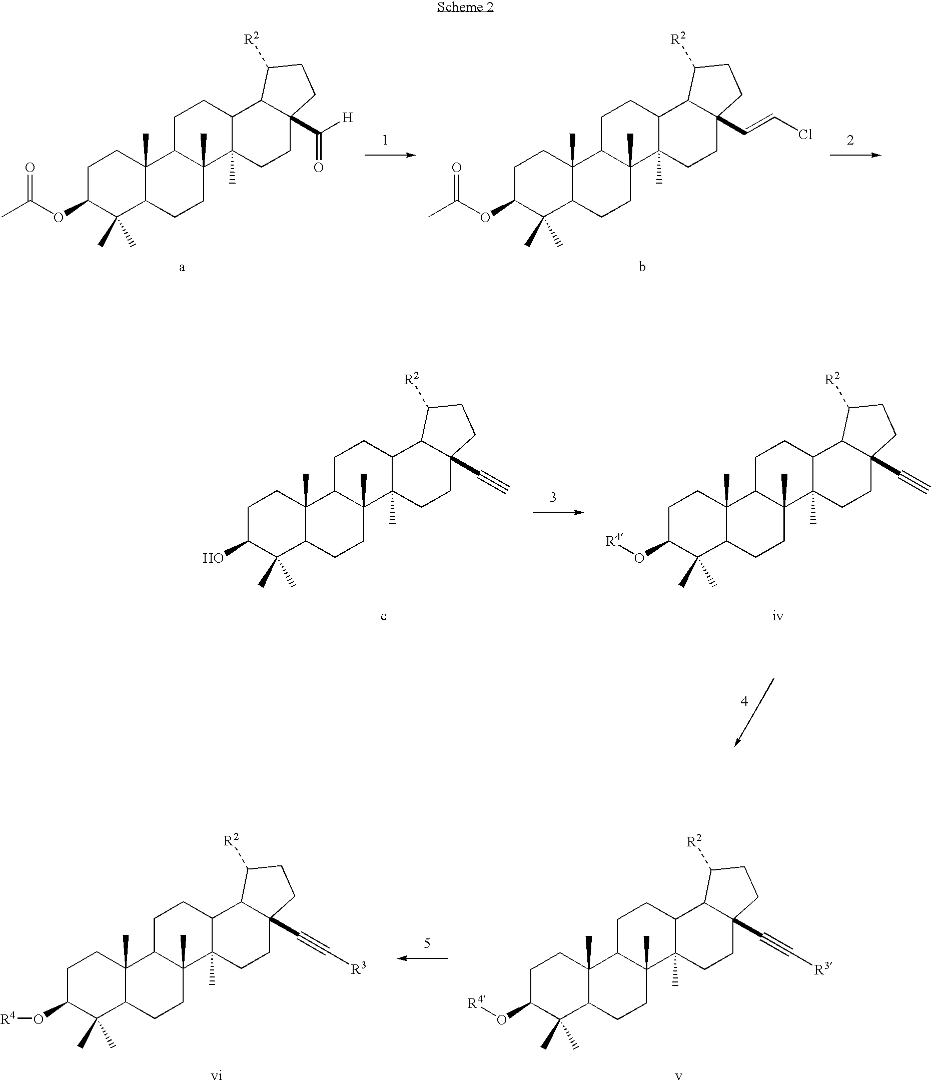

In Scheme 2, the steps of producing Compound c can be same as those in Scheme 1 described above. However, in step 3, Compound c is converted to Compound iv by converting the hydroxyl group at the C-3 position to R^{4′} as defined above, using reagents and under conditions sufficient for the conversion to occur. For example, when R^{4′} is a 3′,3′-dimethylsuccinyl ester, Compound c can be reacted 3-Chlorocarbonyl-2,2-dimethyl-propionic acid methyl ester in the presence of DMAP, DIEA, and DCM. In step 4, Compound iv is reacted with a compound to attach a R^{3′} moiety to the alkynyl group of Compound iv forming Compound v. This can be done, e.g., by reacting Compound iv with an aryl halide or triflate, heteroaryl halide or triflate, carbocyclyl halide or triflate, or heterocyclyl halide or triflate, in a transition metal-catalyzed reaction. In step 5, the R^{3′} and R^{4′} groups in Compound v are converted into the R³ and R⁴ moieties respectively, e.g., by removing any protection group in R^{3′} and R^{4′} thus producing Compound vi. This can be accomplished, e.g., by the hydrolysis of the protection group (e.g., an ester) by saponification in a basic condition.

Thus, the present invention also provides methods of synthesis of compounds as disclosed herein. In various embodiments, a method of synthesis may comprise any one or more steps of Scheme 1 or 2 above.

In one embodiment, a method of making Compound c comprises step 2 of Scheme 1 above, i.e., reacting Compound b with an alkyl lithium in a solvent to produce Compound c. Optionally, the method further comprises of step 1 of Scheme 1 to produce Compound b.

In another embodiment, a method of synthesis comprises step 3, and optionally also Step 2 in Scheme 1 producing Compound d. Step 1 of Scheme 1 may also be included in addition to Steps 2 and 3. In yet another embodiment, a method of making Compound e is provided comprising at least step 4, preferably Steps 3 and 4, also preferably Steps 2, 3 and 4, and more preferably Steps 1, 2, 3 and 4 of Scheme 1 above.

In yet another embodiment, a method of making Compound f is provided comprising at least step 5, preferably Steps 4 and 5, also preferably Steps 3, 4 and 5, or at least Steps 2, 3, 4 and 5, and more preferably Steps 1, 2, 3, 4 and 5 of Scheme 1 above.

In one embodiment, a method of synthesis comprises step 3, and optionally also Step 2, of Scheme 2 producing Compound iv. Step 1 of Scheme 2 may also be included in addition to Steps 2 and 3. In yet another embodiment, a method of making Compound v is provided comprising at least Step 4, preferably Steps 3 and 4, also preferably Steps 2, 3 and 4, and more preferably Steps 1, 2, 3 and 4, of Scheme 2 above.

In yet another embodiment, a method of making Compound vi is provided comprising at least Step 5, preferably Steps 4 and 5, also preferably Steps 3, 4 and 5, or at least Steps 2, 3, 4 and 5, and more preferably Steps 1, 2, 3, 4 and 5 of Scheme 2 above.

The examples below further illustrate the details of the synthesis methods and their application to various compounds of the present invention, as well as the features and characters of such compounds.

### EXAMPLES

### Example 1

### Synthesis of Compounds

### 1. Synthesis of 2,2-Dimethyl-succinic acid 4-[(1R,5aR,5bR,9S,11aR)-3a-(2-carboxy-phenylethynyl)-1-isopropenyl-5a,5b,8,8,11a-pentamethyl-icosahydro-cyclopenta[a]chrysen-9-yl]ester (6)

Synthesis of compound 6 can be accomplished according to the following synthetic route.

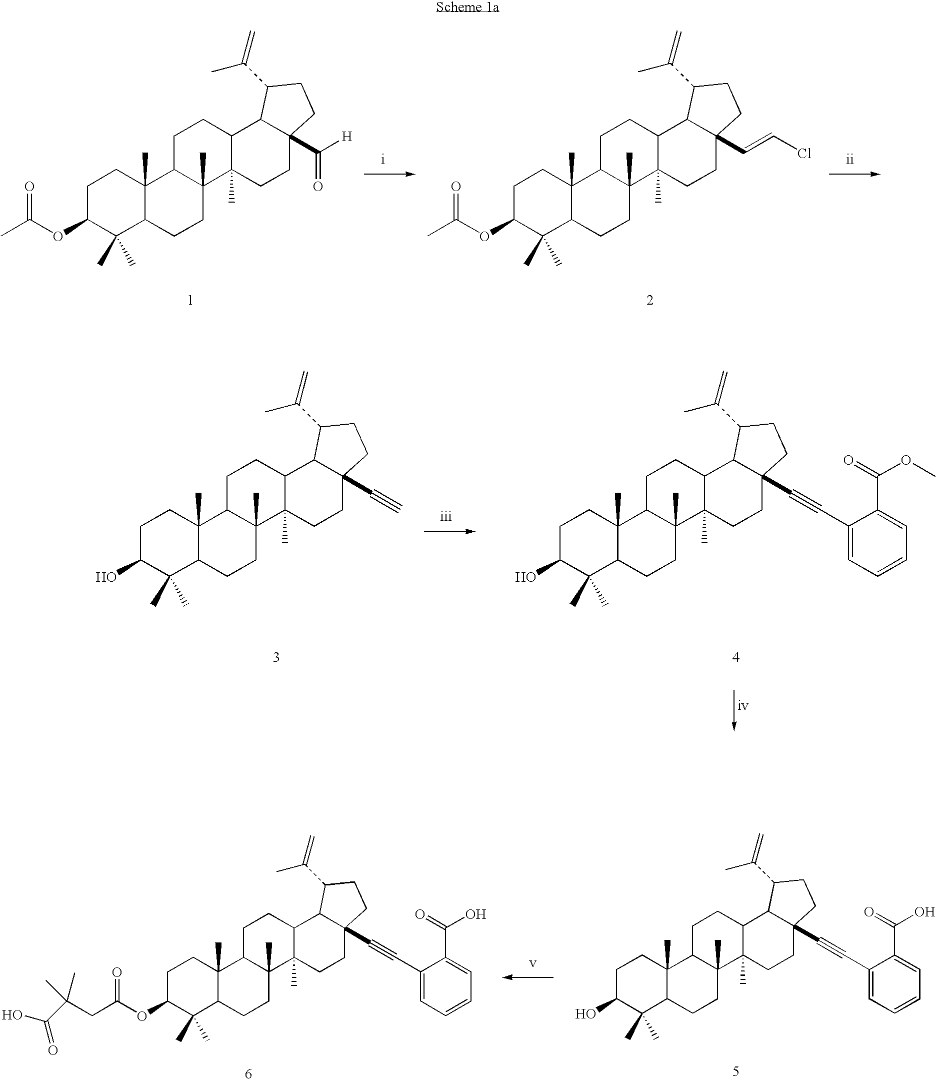

Scheme 1a, Reagents and Conditions: (i) Cl⁻PPh₃⁺CH₂Cl, n-BuLi, HMPA, THF; (ii) MeLi, THF; (iii) Methyl-2-Iodobenzoate, Pd(PPh₃)₂Cl₂, CuI, HN(iPr)₂, THF, 60° C.; (iv) 4 M NaOH, THF, MeOH; (v) 2,2-Dimethylsuccinic anhydride, DMAP, Py, 120° C.

Specifically, 3-acetoxy betulinaldehyde 1 is the commercially available starting material. The key alkynyl intermediate 3 can be obtained from betulinaldehyde 1 via a two step sequence involving Wittig olefination reagents such as (chloromethyl)triphenylphosphonium chloride with bases such as n-butyl lithium in solvents such as THF followed by treatment of the derived vinyl chloride 2 with an alkyl lithium such as methyl lithium in a solvent such as THF at ice-cold temperatures. Coupling of various aryl and heteroaryl halides or triflates such as iodo and bromo derivatives, with compound 3 can be accomplished with various transition metal catalyzed procedures such as with bis(triphenylphosphine)palladium chloride in the presence of cuprous iodide and either secondary or tertiary amines such as diisopropylamine or diisopropylethylamine. Hydrolysis of compound 4 to obtain acid 5 can be accomplished under alkaline conditions such as saponification with 4 M NaOH in solvents such as methanol and THF. Finally, succinylation of acid 5 to obtain final Compound 6 can be carried out with 2,2-dimethyl succinic anhydride in the presence of 4-dimethylaminopyridine in refluxing solvents such as pyridine. The final compound was purified to remove undesired minor C-3 regioisomer after succinylation reaction.

Acetic acid (1R,5aR,5bR,9S,11aR)-3a-((E)-2-chloro-vinyl)-1-isopropenyl-5a,5b,8,8,11a-pentamethyl-icosahydro-cyclopenta[a]chrysen-9-yl ester (2):

To a suspension of (chloromethyl)triphenylphosphonium chloride (1.8 g, 5 mmol) in anhydrous THF (50 mL) at ice-cold temperatures was added dropwise n-butyl lithium (3.12 mL, 5 mmol) during 15 minutes. HMPA (0.516 mL, 3.09 mmol) was added and the mixture was allowed to stir for 20 minutes. 3-Acetoxy betulinaldehyde (500 mg, 1.03 mmol) in THF (5 mL) was added gradually over 10 minutes and the reaction mixture was allowed to stir at room temperature overnight. The reaction mixture was diluted with ethyl acetate (50 mL) before it was quenched with aqueous 1 N hydrochloric acid (10 mL) and extracted. The organic layer was collected and dried over anhydrous sodium sulfate. Evaporation of the solvent furnished a solid residue that was purified by silica gel chromatography using ethyl acetate and hexane as eluents providing vinyl chloride 2 as white powder (250 mg). Structure of the product was confirmed with proton NMR.

(1R,5aR,5bR,9S,11aR)-3a-Ethynyl-1-isopropenyl-5a,5b,8,8,11a-pentamethyl-icosahydro-cyclopenta[a]chrysen-9-ol (3):

To an ice-cold solution of vinyl chloride 2 (250 mg, 0.5 mmol) in THF (15 mL) was added drop-wise methyl lithium (1.6 mL, 2.5 mmol) during 15 minutes. The reaction was allowed to warm to room temperature and stir for 24 h, cooled again with an ice-cold water bath and quenched by the drop-wise addition of aqueous 1 N hydrochloric acid (10 mL) during 10 minutes. The solution was extracted with ethyl acetate (2×25 mL) and the organic layer was washed with water, brine and dried over anhydrous sodium sulfate. Alkyne derivative 3, obtained as a white powder (155 mg), was used as such in the next step without further purification. The structure of the product was confirmed by proton NMR.

2-((1R,5aR,5bR,9S,11aR)-9-Hydroxy-1-isopropenyl-5a,5b,8,8,11a-pentamethyl-icosahydro-cyclopenta[a]chrysen-3a-ylethynyl)-benzoic acid methylester (4):

To a magnetically stirred solution of alkyne 3 (100 mg, 0.22 mmol) in anhydrous THF (15 mL) under a nitrogen atmosphere was added methyl-2-iodobenzoate (60 mg, 0.22 mmol), bis(triphenlphosphine)palladium chloride (14 mg, 0.011 mmol), cuprous iodide (4 mg, 0.011 mmol) and diisopropylamine (100 μL, 0.33 mmol) and the mixture was heated at 60° C. during 4 hrs. The reaction mixture was quenched with aqueous 1 N hydrochloric acid (5 mL) and extracted with ethyl acetate (2×20 mL). The organic layer was washed with water, brine and dried over anhydrous sodium sulfate. Evaporation of the solvent yielded a yellow residue that was purified by silica gel chromatography using ethyl acetate and hexane as eluent providing alkynyl benzoate 4 as a white powder (70 mg). Structure of the product was confirmed by proton NMR and mass spectroscopy.

2-((1R,5aR,5bR,9S,11aR)-9-Hydroxy-1-isopropenyl-5a,5b,8,8,11a-pentamethyl-icosahydro-cyclopenta[a]chrysen-3a-ylethynyl)-benzoic acid (5):

To a stirred solution of alkynyl benzoate 4 (70 mg, 0.13 mmol) in THF (5 mL) and MeOH (5 mL) was added 4 M aqueous sodium hydroxide (1 mL) and the resultant mixture was stirred at room temperature for 2 h. Solvent was evaporated and the residue was acidified with aqueous 6 N HCl (5 mL) and extracted with ethyl acetate (2×20 mL). The organic layer was washed with water, brine and dried over anhydrous sodium sulfate. Evaporation of the solvent furnished alkynyl benzoic acid 5 (55 mg) as a white solid, the structure of which was confirmed by proton NMR and mass spectroscopy.

2,2-Dimethyl-succinic acid 4-[(1R,5aR,5bR,9S,11aR)-3a-(2-carboxy-phenylethynyl)-1-isopropenyl-5a,5b,8,8,11a-pentamethyl-icosahydro-cyclopenta[a]chrysen-9-yl]ester (6):

To a magnetically stirred solution of alkynyl benzoic acid 5 (50 mg, 0.09 mmol) in anhydrous pyridine (2 mL) under a nitrogen atmosphere was added 2,2-dimethyl succinic anhydride (55 mg, 0.45 mmol) and 4-dimethylaminopyridine (55 mg, 0.45 mmol) and the mixture was heated at 105° C. for 24 h. The reaction was cooled, solvent was rotary evaporated, and the residue was dissolved in CH₂Cl₂ (20 mL) and washed with aqueous 1 N HCl (10 mL). The organic layer was washed with water, brine and dried over anhydrous sodium sulfate. Evaporation of the solvent and the purification of the residue by silica gel chromatography employing ethyl acetate and hexane as eluent furnished compound 6 as white solid (25 mg) for which the structure was established based on proton NMR and mass spectroscopy.

Synthesis of compound 6 can also be accomplished according to the following synthetic route:

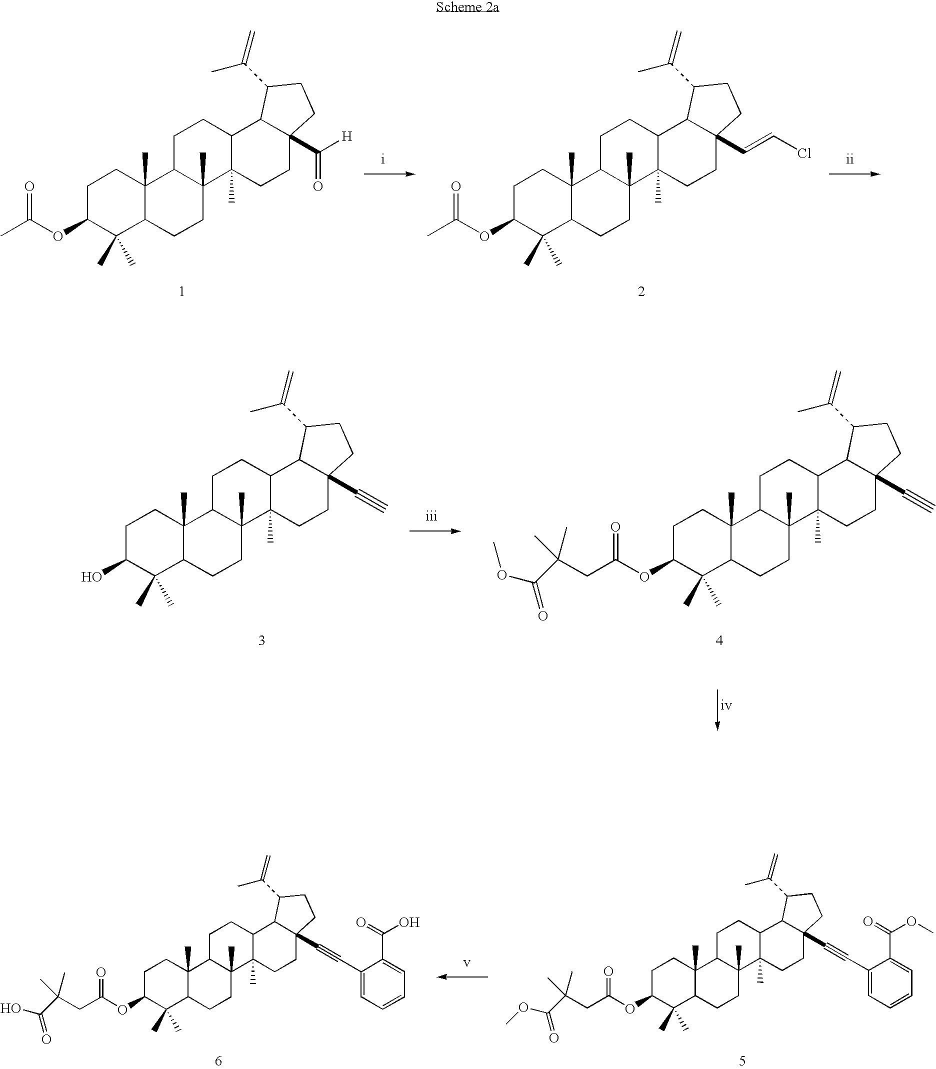

Scheme 2a, Reagents and Conditions: (i) Cl⁻PPh₃⁺CH₂Cl, n-BuLi, HMPA, THF; (ii) MeLi, THF; (iii) 3-Chlorocarbonyl-2,2-dimethyl-propionic acid methyl ester, DMAP, DIEA, DCM, 60° C., 4-6 hr; (iv) Methyl-2-Iodobenzoate, Pd(PPh₃)₂Cl₂, CuI, HN(iPr)₂, THF, 60° C.; (v) 4 M NaOH, THF, MeOH.

Specifically, alkynyl intermediate 3 was prepared as described in Scheme 1a from 3-acetoxy betulinaldehyde 1 via a two-step sequence. Alkyne intermediate 3 was then treated with 2 to 3 equivalents of 3-chlorocarbonyl-2,2-dimethyl-propionic acid methyl ester, with base such as dimethylamino pyridine and diisopropyl ethylamine, in solvent such as dichloromethane or THF either at room temperature for 8-14 hours or 60° C. for 2-6 hours. Coupling of various aryl and heteroaryl halides such as iodo and bromo derivatives, with compound 4 were accomplished with various transition metal catalyzed procedures such as with bis(triphenylphosphine)palladium chloride in the presence of cuprous iodide and either secondary or tertiary amines such as diisopropylamine or diisopropylethylamine to obtain compound 5. Hydrolysis of compound 5 to obtain acid 6 were accomplished under alkaline conditions such as saponification with 4 M NaOH in solvents such as methanol and THF. The intermediate compound 4 in Scheme 2a was isolated and saponified with 4M NaOH (aqueous) in methanol at room temperature and characterized:

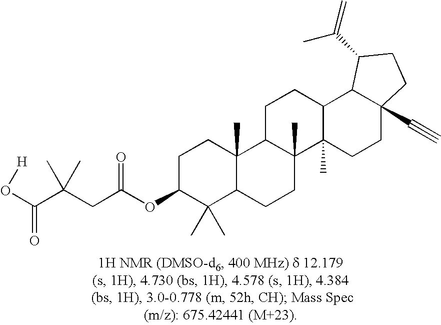

### 2. Synthesis of Some Other Compounds

Compounds 7-14, 17, 19-27, 29-36, 38, 44, 46, 47, and 50-54 of Table 2 below were prepared according to general Scheme 1 from the respectively appropriate aryl halide derivative using the reagents and conditions detailed above in Scheme 1a. Similarly, compounds 15, 16 and 40 of Table 2 were derived from dihydro betulinaldehyde, prepared according to Scheme 3 below by using the appropriate aryl halide derivative, reagents and conditions detailed above for compound 6.

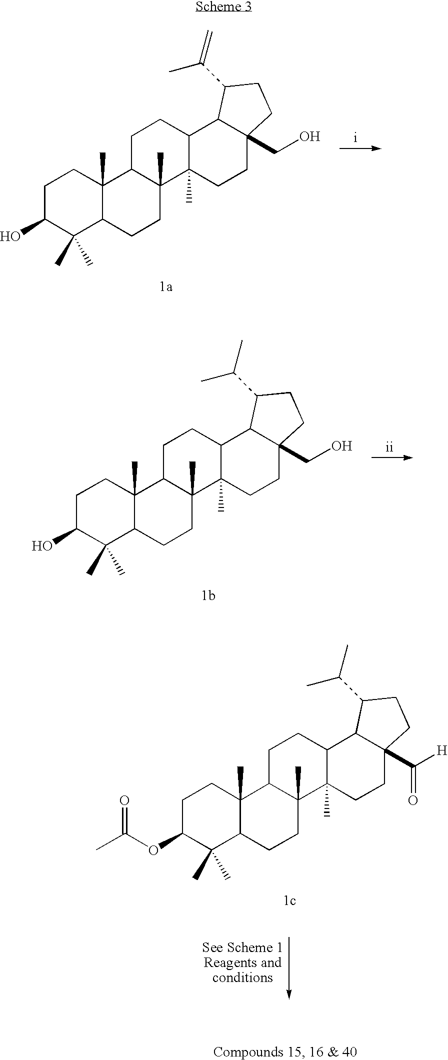

Reagents and Conditions: (i) H₂, PtO₂/C, MeOH; (ii) see U.S. Pat. No. 6,232,481.

(1S,3aS,5aR,5bR,9S,11aR)-3a-Hydroxymethyl-1-isopropyl-5a,5b,8,8,11a-pentamethyl-icosahydro-cyclopenta[a]chrysen-9-ol (1b):

To a solution of betulin (1a) (5 g, 11.2 mmol) in AcOH (200 mL) and THF (200 mL) was added PtO₂ (100 mg) and the reaction mixture was allowed to stir under H₂ gas (50 psi) for 8 hours at ambient temperature. The catalyst was filtered over Celite and was washed with THF. The solvent was evaporated to provide dihydro betulin (1b) (5 g) which was dried overnight under vacuum and used as such in the next step.

Compound 39 of Table 2 below was prepared according to general Scheme 1 from the appropriate aryl halide derivative using the reagents and conditions detailed above in Scheme 1a, except 3,3′-dimethyl-glutaryl anhydride was used in lieu of 3′3′-dimethyl-succinyl anhydride.

### 3. Synthetic of Compounds 18 and 37:

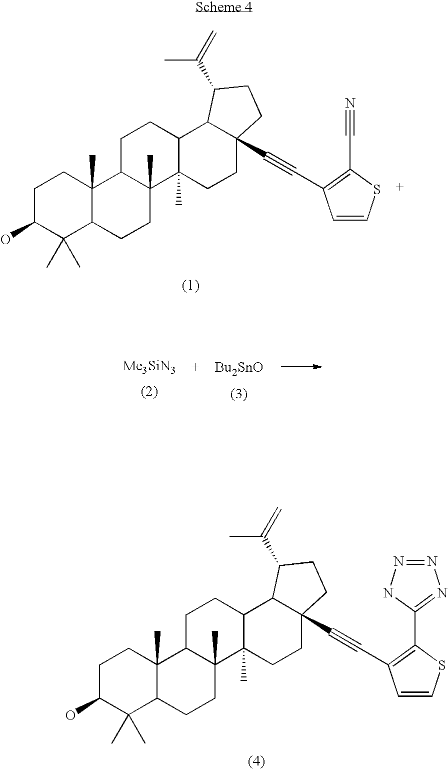

Compound (1) was prepared in a similar way as described in Scheme 1, using 3-Bromo-thiophene-2-carbonitrile as a starting material for coupling reaction. To a toluene (1.5 ml) solution of nitrile (1) (2.4 mmols, 1 eq), trimethylsilyl azide (2) (4.8 mmols, 2 eq), and di-n-butynl oxide (3) (2.4 mmols, 0.1 eq) were added (as shown in Scheme 3) in a microwave. The reaction mixture was heated to 110° C. for 2 hrs. The reaction mixture was concentrated then extracted with dichloromethane and water to give a crude product that was used without further purification. The tetrazole derivative (4) was converted into final Compound 18 employing similar conditions as described in Scheme 1.

Synthesis of Compound 37 was done using the same procedure in Scheme 4 except with the starting material 4-Fluoro-2-((1R,3aS,5aR,5bR,9S,11aR)-9-hydroxy-1-isopropenyl-5a,5b,8,8,11a-pentamethyl-icosahydro-cyclopenta[a]chrysen-3a-ylethynyl)-benzonitrile.

### 4. Synthesis of Compound 28:

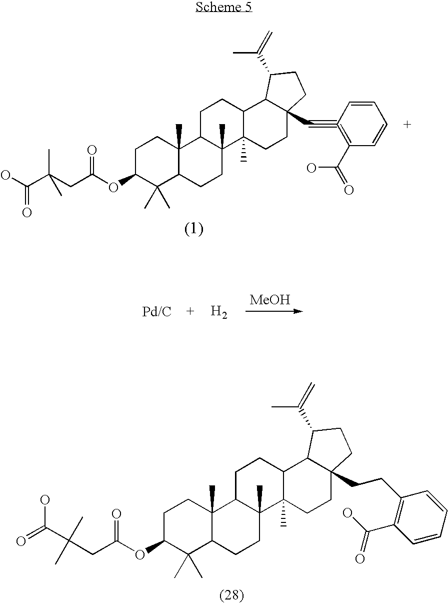

Added diacid (1) (Scheme 5) (0.263 mmols, 1 eq) to a round bottom flask containing palladium on carbon (0.018 g, 10% by weight) and 10 ml of dry methanol. The reaction mixture was stirred at room temperature for 18 hours under hydrogen at balloon pressure to give tetrahydro derivative (28) with approximately 15% yield. Purification was performed by reverse phase HPLC.

### 5. Synthesis of Compounds 41, 45, and 49:

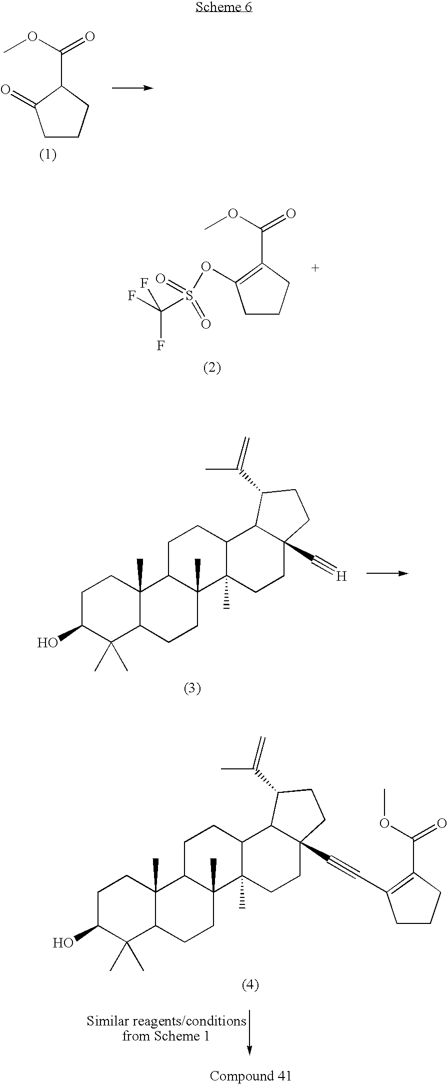

To a solution of methyl 2-oxocyclopentocarboxylate (1) (3 mL, 24.16 mmol) in DCM (60 mL, 0.4M) was added DIPEA (21 mL, 102 mmol, 5 equiv.) at −78° C. The mixture was stirred for 10 minutes during which Tf₂O (3.71 mL, 29 mmol, 1.2 equiv.) was added in a drop wise fashion, followed by slow warming to room temperature overnight. The reaction was monitored by TLC and upon completion, the mixture was washed with water (50 mL) and 10% aq. citric acid (100 mL twice). The combined organic layers were dried with Na₂SO₄, concentrated under vacuum. The residue was purified by silica gel flash chromatography (10% Ethylacetate and Hexane) to produce the product (2) (5.39 g, 81% yield).

To a magnetically stirred solution of the alkyne (3) (0.3 g, 0.687 mmol) in anhydrous THF (4.5 mL, 0.1M) under a nitrogen atmosphere was added (2) (565 mg, 2.06 mmol, 3 equivalents), bis(triphenylphosphine)palladium chloride (48 mg, 0.069 mmol, 0.1 equiv.), cuprous iodide (13 mg, 0.069 mmol, 0.1 equivalent) and diisopropylamine (386 μL, 2.75 mmol, 4 equivalents). The mixture was stirred at room temperature overnight. Upon completion, the reaction mixture was quenched with aqueous 1N hydrochloric acid (3 mL) and the aqueous layer was extracted with ethyl acetate (4×15 mL). The organic layer was washed with water, brine and dried over anhydrous sodium sulfate. Evaporation of the solvent yielded a yellow residue that was purified through silica gel flash chromatography using 0-20% ethyl acetate and hexane as eluent. The product (4) is a white solid (282 mg, 73% yield). Structure of the product was confirmed by ¹H NMR. The compound (4) in Scheme 6 was further elaborated to the final Compound 41 using reagents and under conditions described in Scheme 1.

Compound 45 and 49 were synthesized using the same procedure in Scheme 6 described above for Compound 41, except using appropriate different starting materials.

### 6. Synthesis of Compound 42 and 43.

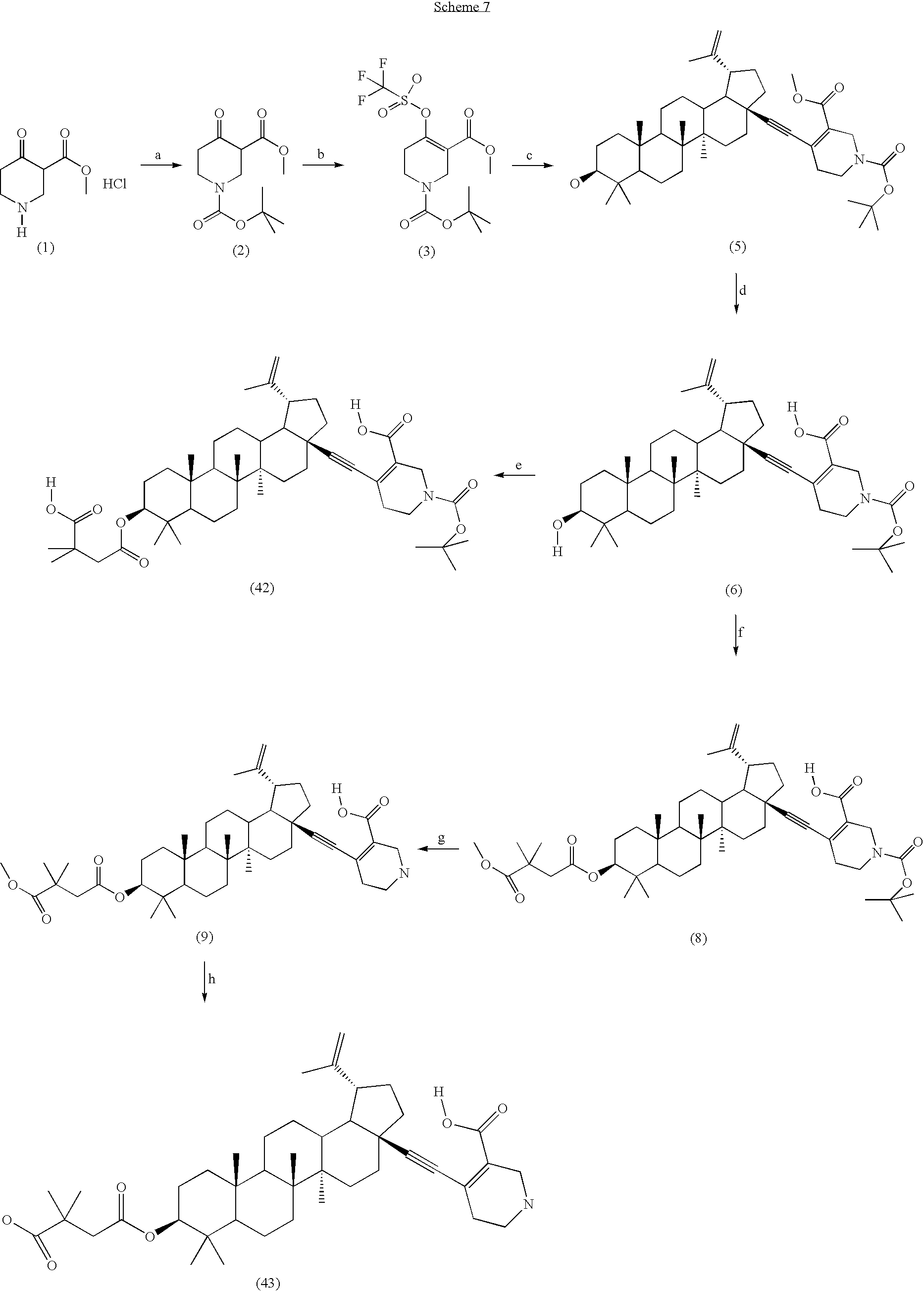

The compound (2) in Scheme 7 was prepared using reported procedure J. Org. Chem. 1992, 57, 2794.

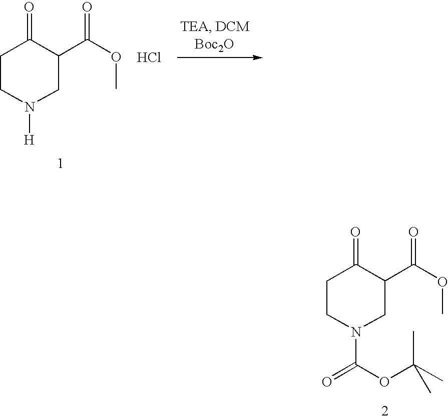

To a stirred slurry of 1 in Scheme 7 (1 g, 10.33 mmol, 1 equiv.) in DCM (10.5 mL, 0.5M) and TEA (32 mL, 25.8 mmol, 2.5 equiv.) at 0° C. was added Boc₂O (1.25 g, 11.4 mmol, 1.1 equiv.) as a solid. The solution was stirred at that temperature for 1 hour and at room temperature for an additional hour. The mixture was poured in ice cold HCl (2N, 50 mL) and extracted with DCM twice. The extract were combined and washed with saturated aq. NaHCO₃ solution. The product was confirmed by NMR analysis. The residue was purified by flash chromatography (20% Ethylacetate and Hexane as eluant). (1.28 g, 48% yield).

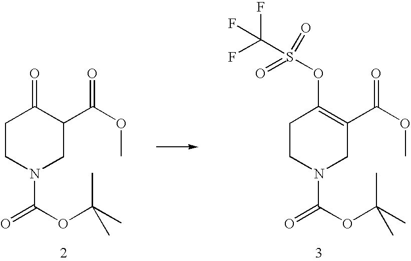

To a solution of 2 (1.28 g, 4.98 mmol, 1 equiv.) in DCM (18 mL, 0.3M) was added DIPEA (4.3 mL, 25 mmol, 5 equiv.) at −78° C. The mixture was stirred for 10 mins during which Tf₂O (0.77 mL, 6 mmol, 1.2 equiv.) was added in a dropwise fashion, followed by slow warming to room temperature overnight. The mixture was washed with water (50 mL) and 10% aq. citric acid (100 mL twice). The organic layer was dried, concentrated under vacuum, purified by flash chromatography (20% Ethylacetate and Hexane) to produce (3) (1.46 g, 77% yield).

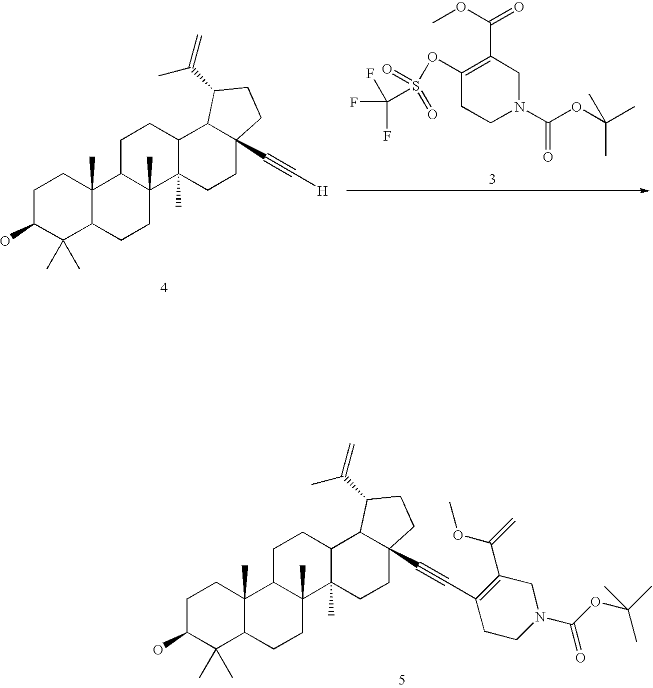

To a magnetically stirred solution of alkyne 4 (0.2 g, 0.458 mmol, 1 equiv.) in anhydrous THF (5 mL, 0.1M) under a nitrogen atmosphere was added 3 (533 mg, 1.37 mmol, 3 equiv.), bis(triphenlphosphine)palladium chloride (32 mg, 0.046 mmol, 0.1 equiv.), cuprous iodide (9 mg, 0.046 mmol, 0.1 equiv.) and diisopropylamine (257 μL, 1.83 mmol, 4 equiv.) and the mixture was stirred at room temperature, overnight. The reaction was monitored by TLC, and upon completion the reaction mixture was quenched with aqueous 1 N hydrochloric acid (2 mL). The aqueous layer was extracted with ethyl acetate (4×15 mL). The combined organic layer were washed with brine and dried over anhydrous sodium sulfate. Evaporation of the solvent yielded a residue that was purified by silica gel flash chromatography (40 g) using ethyl acetate and hexane as eluent (0-20%) providing the product 5 as a brown powder (195 mg, 63% yield).

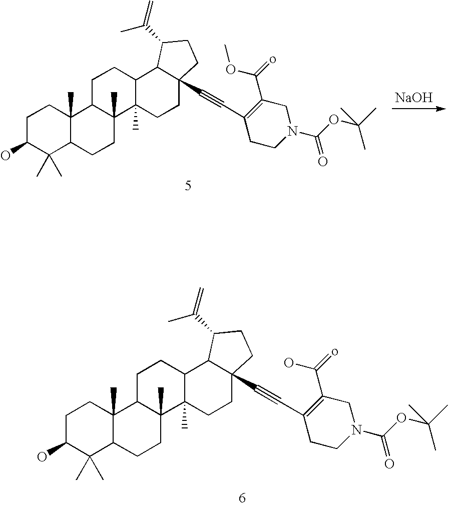

To a stirred solution of 5 (391 mg, 0.579 mmol, 1 equiv.) in THF (5 mL, 0.12M) and MeOH (5 mL, 0.12M) was added 4N aqueous sodium hydroxide (1.44 mL, 10 equiv.) and the resultant mixture was heated to 50° C. for 3 hours to get the reaction to completion. The solvent was evaporated and the solid residue thus obtained was suspended in aqueous 1 N HCl (5.8 mL) to reach neutral pH. The suspension was extracted with ethyl acetate and sonicated to solubilize all white solids. The organic phase was then filtered through a pad of celite and the solvent rotovaped to provide acid 6 as an off white solid (352 mg, 92%) that was dried overnight and used in the next step without further purification.

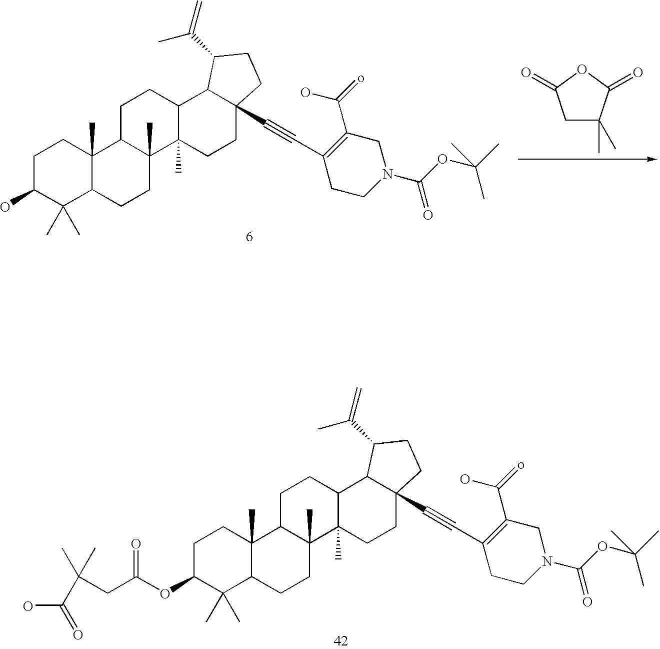

To a magnetically stirred solution of 2,2-dimethyl succinic anhydride (53 mg, 0.41 mmol, 5 equiv.), 6 (54.8 mg, 0.083 mmol, 1 equiv.) in anhydrous pyridine (1 mL) was added 4-dimethylaminopyridine (50 mg, 0.41 mmol, 5 equiv.) at room temperature. After 30 mins, the solvent was evaporated and the residue was re-dissolved in anhydrous pyridine (1 mL). The solution was heated at 120° C. for 24 hours under a nitrogen atmosphere. Upon reaction completion, the reaction was cooled, the solvent rotary evaporated and the residue was suspended in 4 N HCl (1 mL), sonicated and stirred at room temp for 2 hours. The suspension was extracted with ethyl acetate and the solvent dried under vacuum. The product 42 thus obtained was further purified by reverse phase HPLC to yield a white solid (29 mg, 44%) for which the structure was established based on proton NMR and mass spectroscopy.

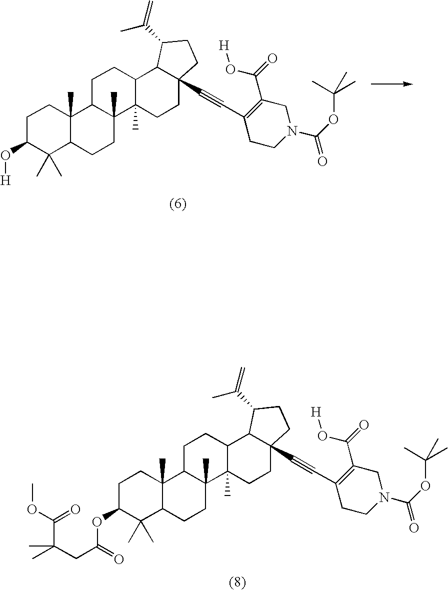

To a magnetically stirred solution of monoester of 2,2-dimethyl succinic ester (465 mg, 2.9 mmol, 5.4 equiv.) in anhydrous DCM (60 mL, 0.05M) was added oxalyl chloride (982 μL, 11.6 mmol, 22 equiv.) at room temperature, followed by the addition of DMF (catalytic). Additional DMF (1 drop) was added after 60 minutes. The solvent was evaporated after 2.5 hours and the pale yellow oil was taken in anhydrous DCM (5 mL). This solution was added to the pre-mixed solution of the alkyne 6 (352 mg, 0.531 mmol, 1 equiv.), DMAP (355 mg, 2.9 mmol, 5.4 equiv.), DIPEA (495 μL, 2.9 mmol, 5.4 equiv.) in DCM (50 mL, 0.1M) at 0° C. The addition was slow and the mixture was allowed to warm up to room temperature overnight under nitrogen atmosphere.

Upon completion, the reaction was cooled, and quenched with HCl (1N). The aqueous layer was extracted twice with DCM. The combined organic phases were dried with sodium sulfate and rotovaped under vacuum. The residue thus obtained was further purified by silica gel flash chromatography (0-10% EA/Hex.) to yield a white solid 8 (295.4 mg, 70% yield) for which the structure was established based on proton NMR and mass spectroscopy.

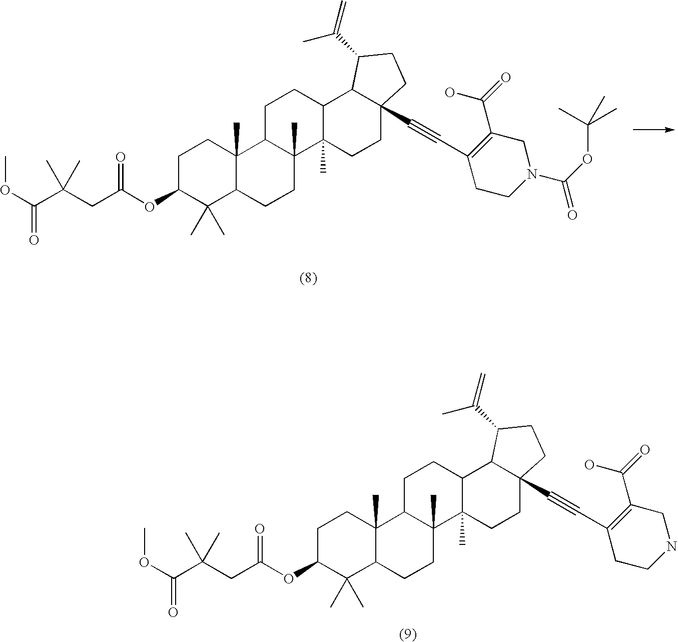

To a magnetically stirred solution of the Boc-protected amine 8 (242 mg, 0.3 mmol, 1 equiv.) in anhydrous DCM (10 mL, 0.03M) was added TFA (1 mL) at room temperature. The reaction was completed after 2 hours and the solvent was evaporated under vacuum. The oily residue obtained was further purified by preparative TLC (15% MeOH/CH₂Cl₂) to yield a white solid 9 (95 mg, 45% yield) for which the structure was established based on proton NMR and mass spectroscopy.

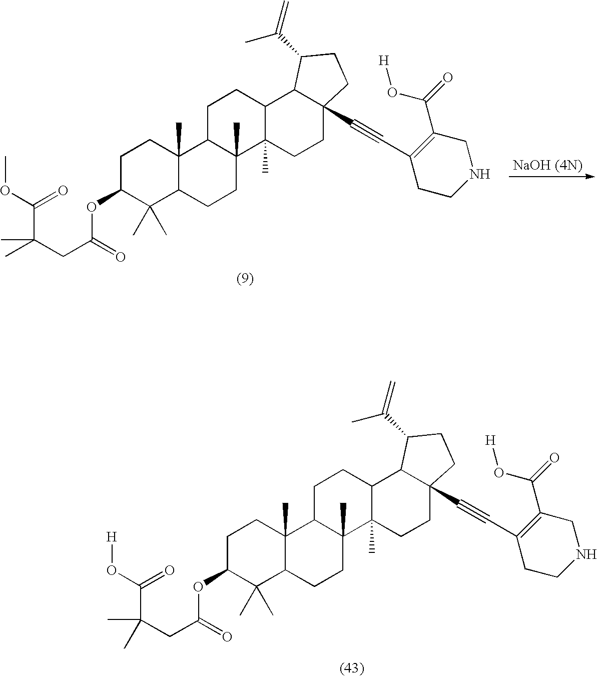

To a magnetically stirred solution of ester 9 (30 mg, 0.043 mmol, 1 equiv.) in THF (0.2 mL) and MeOH (0.2 mL) was added an aqueous NaOH (4N, 1 mL) and the resultant mixture was stirred at room temperature overnight. Upon completion, the reaction was quenched with aqueous HCl (1N, 4.3 mL) and the solvent was evaporated in vacuo. The solid residue thus obtained was purified by reverse phase HPLC to provide 43.

7. Synthesis of Compound 48.

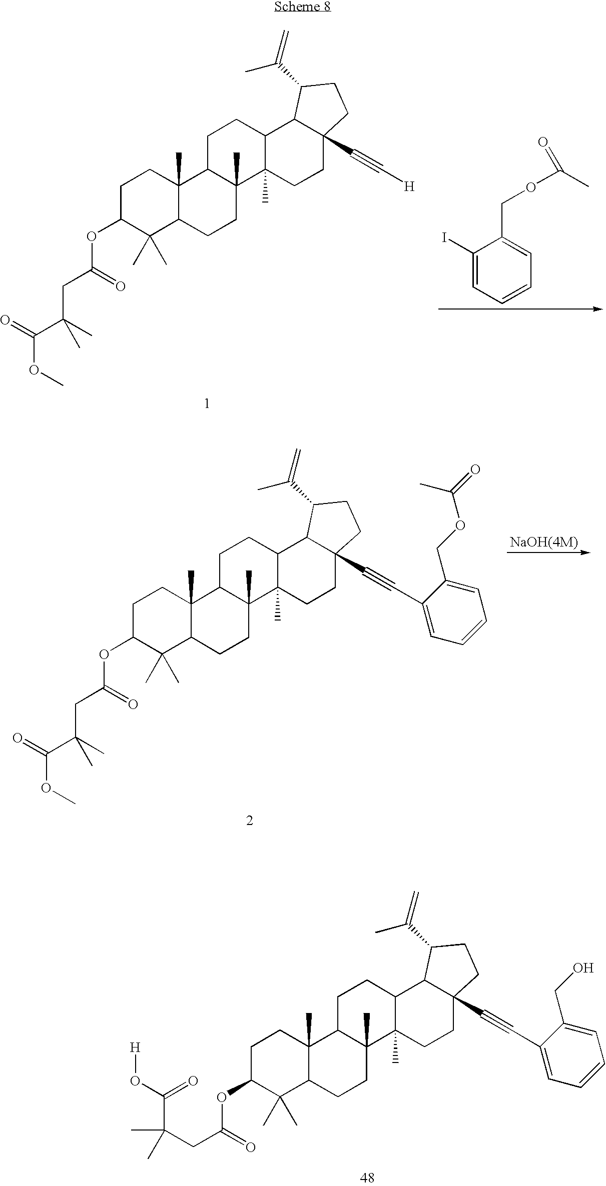

To a magnetically stirred solution of alkyne 1 in Scheme 8 (0.3 g, 0.519 mmol, 1 equiv.) in anhydrous THF (5 mL, 0.1M) under nitrogen atmosphere was added iodobenzene (430 mg, 2.08 mmol, 4 equiv.), bis(triphenlphosphine)palladium chloride (37 mg, 0.052 mmol, 0.1 equiv.), cuprous iodide (10 mg, 0.052 mmol, 0.1 equiv.) and diisopropylamine (292 μL, 2.08 mmol, 4 equiv.). The mixture was heated to 50° C. and stirred overnight.

Upon completion, the reaction mixture was quenched with aqueous hydrochloric acid (1N, 3 mL) and the aqueous layer was extracted with ethyl acetate (4×15 mL). The organic layer was washed brine and dried over anhydrous sodium sulfate. Evaporation of the solvent yielded a residue that was purified by silica gel flash chromatography (40 g) using ethyl acetate and hexane as eluent (0-10%) providing the product 2 as a white powder (273 mg, 73% yield). Structure of the product was confirmed by proton NMR.

To a stirred solution of alkynyl derivative 2 (50 mg, 0.069, 1 equiv.) in THF/MeOH (1:1, 1 mL) at room temperature was added aqueous NaOH (4N, 1.75 mL, 100 equiv.). The reaction went to completion after overnight stirring and was quenched with HCl (4N, 2 mL). The organic solvents were evaporated under vacuum and the residue stirred in water for 2 hours before filtration. The solid product 48 was purified by reverse phase HPLC and characterized by ¹H NMR and mass spectrometry.

8. Synthesis of Compound 55.

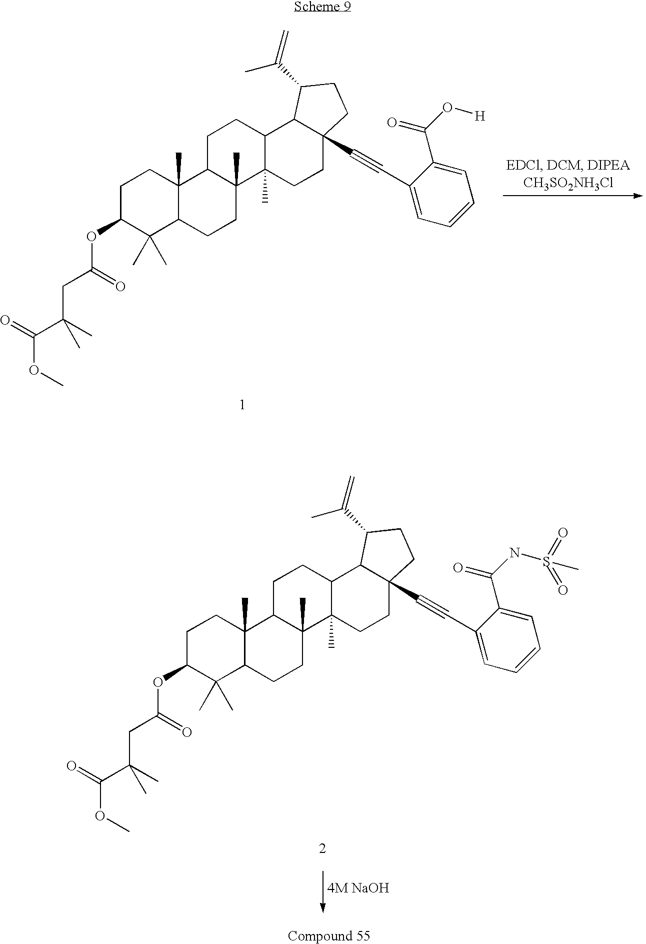

To a magnetically stirred solution of benzoic acid 1 in Scheme 9 (100 mg, 0.143 mmol, 1 equiv.) in anhydrous DCM (3 mL, 0.05M) under a nitrogen atmosphere was added methanesulfonamide (38 mg, 0.286 mmol, 2 equiv.), EDCI (55 mg, 0.286 mmol, 2 equiv.), DMAP (39 mg, 0.314 mmol, 2.2 equiv.) and DIPEA (54 μL, 0.314 mmol, 2.2 equiv.) and the mixture was stirred overnight at room temperature. Upon completion, the reaction mixture was quenched with aqueous hydrochloric acid (1N, 1 mL) and the aqueous layer was extracted with ethyl acetate (5 mL twice). The organic layer was washed with water, brine and dried over anhydrous sodium sulfate. Evaporation of the solvent yielded a residue that was purified by silica gel preparative TLC using ethyl acetate and hexane as eluent 0-40% providing the product 2 (79 mg, 71% yield). Structure of the product 2 was confirmed by proton ¹H NMR. The ester in product 2 was hydrolyzed to obtain Compound 55 using 4 M NaOH as described in the synthesis for Compound 48.

The structures of the exemplary compounds made and characterization thereof are provided in Table 2 below.

TABLE 2

| Compound | | ¹H-NMR (400 MHz) δ |
|---|---|---|
| No. | Structure | LC-MS (ESI) |
| 6 | 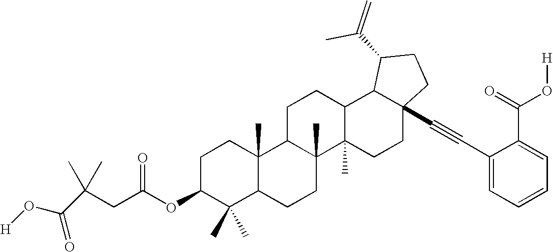 | (CDCl₃) 8.05 (dd, 1 H, J = 7.6 and 0.8 Hz), 7.55-7.47 (m, 2 H), 7.35 (d of t, 1 H, J = 7.6 and 1.6 Hz), 4.75 (br s, 1 H), 4.60 (br s, 1 H), 4.50 (t, 1 H, J = 15.6 Hz), 2.8-0.7 (m, 53 H). 683.5 (M − 1) |
| 7 | 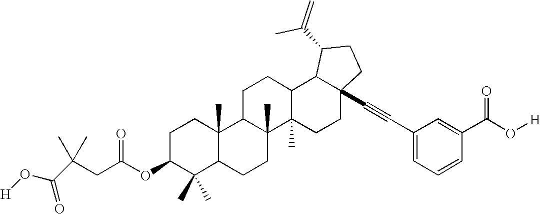 | (CDCl₃) 8.10 (t, J = 1.5 Hz, 1 H), 7.98 (dt, J = 1.3, 7.8 Hz, 1 H), 7.63 (dt, J = 1.4, 7.5 Hz, 1 H), 7.40 (t, J = 7.5, 1 H), 4.74 (s, 1 H), 4.61 (s, 1 H), 4.5 (t, J = 8.3 Hz, 1 H), 2.64 (m, 4 H), 2.17 (p, J = 9.0 Hz, 1 H), 2.04 (m, 2 H), 1.94 (t, J = 13.4 Hz, 1 H) 1.78-0.81 (m, 43 H); 707.4216 (M + Na). |
| 8 | 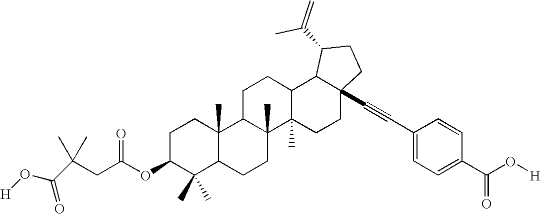 | (CDCl₃) 8.01 (d, 2 H, J = 8 Hz), 7.48 (d, 2 H, J = 8 Hz), 4.73 (br s, 1 H), 4.61 (br s, 1 H), 4.52-4.46 (m, 1 H), 2.8-0.7 (m, 63 H). 683.3 (M − 1) |
| 9 | 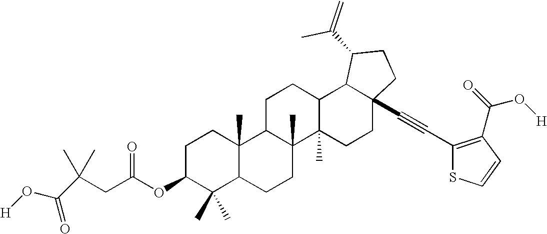 | (DMSO-d₆) 7.36 (bs, 1 H, Ar), 7.26 (bs, 1 H, Ar), 4.72 (s, 1 H, CH═), 4.58 (s, 1 H, CH═), 4.37 (bs, 1 H), 2.30- 0.64 (m, 51 H, CH); 689.4 (M − 1). |
| 10 | 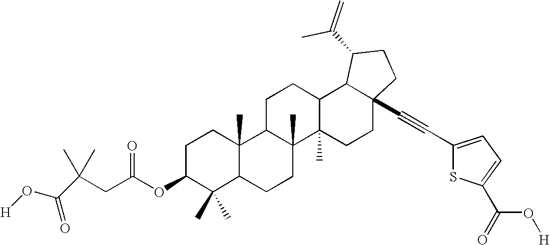 | (DMSO-d₆) 7.60 (bs, 1 H, Ar), 7.21 (bs, 1 H, Ar), 4.74 (bs, 1 H, CH═), 4.59 (bs, 1 H, CH═), 4.36 (bs, 1 H), 2.30-0.64 (m, 51 H, CH); 689.4 (M − 1). |
| 11 | 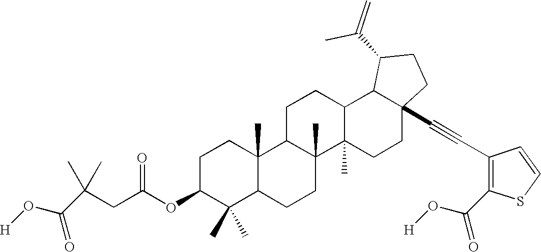 | (DMSO-d₆) 7.79 (d, 1 H, J = 5.6 Hz), 7.11 (d, 1 H, J = 5.6 Hz), 4.72 (br s, 1 H), 4.57 (br s, 1 H), 4.40-4.30 (m, 1 H), 2.8-0.7 (m, 55 H). 689.5 (M − 1). |
| 12 | 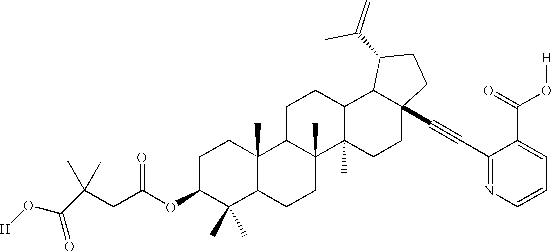 | (DMSO-d₆) 8.66 (dd, J = 4.8 Hz, J = 1.6 Hz, J = 1.6 Hz, 1 H, Ar), 8.14 (dd, J = 8 Hz, J = 2 Hz, J = 1.6 Hz, 1 H, Ar), 7.44 (dd, J = 7.6 Hz, J = 4.8 Hz, J = 5.2 Hz, 1 H, Ar), 4.74 (s, 1 H, CH═), 4.59 (s, 1 H, CH═), 4.37 (dd, J = 11.2 Hz, J = 4.8 Hz, J = 5.2 Hz, 1 H), 2.76-0.64 (m, 51 H, CH); 686.41315 (M + 1). |
| 13 | 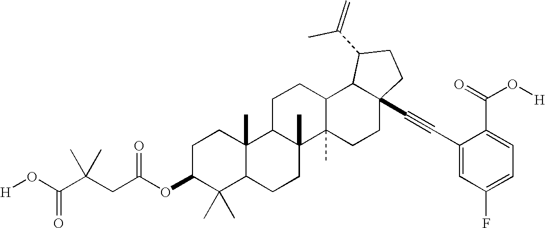 | (DMSO-d₆) 8.07 (dd, J = 8.8 Hz, J = 6 Hz, J = 5.6 Hz, 1 H, Ar), 7.21 (dd, J = 9.2 Hz, J = 2.8 Hz, J = 2.8 Hz, 1 H, Ar), 7.06-7.01 (m, 1 H, Ar), 4.752 (s, 1 H, CH═), 4.604 (s, 1 H, CH═), 4.52-4.48 (m, 1 H), 2.76-0.64 (m, 51 H, CH); 703.4447 (M + 1). |
| 14 | 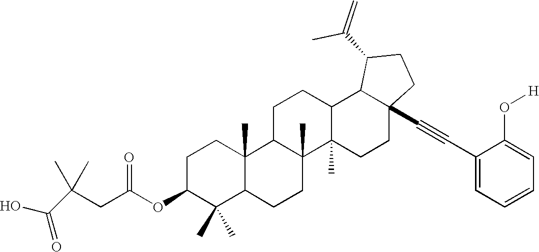 | (CDCl₃) 7.32 (d, J = 7.1 Hz, 1 H), 7.22 (t, J = 7.1 Hz, 1 H), 6.95 (d, J = 8.3 Hz, 1 H), 6.87 (t, J = 7.1 Hz, 1 H), 4.73 (s, 1 H), 4.62 (s, 1 H), 4.49 (m, 1 H), 2.63 (m, 3 H), 2.17 (m, 2 H), 1.95 (m, 5 H), 1.79-1.16 (m, 27 H), 1.08 (s, 3 H), 0.96 (s, 3 H), 0.90- 0.71 (m, 8 H); 675.6783 (M + Na). |
| 15 | 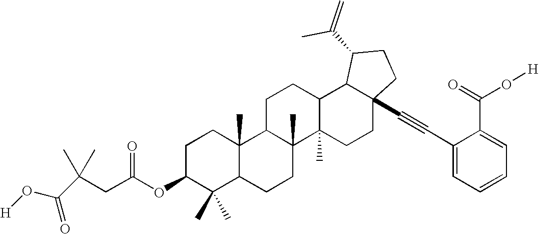 | (CDCl₃) 8.06 (d, 1 H, J = 8 Hz), 7.54-7.40 (m, 2 H), 7.33 (t, 1 H, J = 7.2 Hz), 4.54-4.45 (m, 1 H), 2.83- 2.43 (m, 2 H), 2.13-0.6 (m, 55 H) 685.5 (M − 1) |
| 16 | 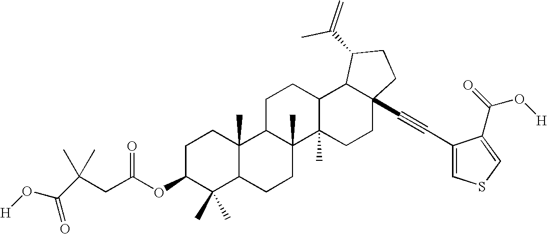 | (DMSO-d₆) 8.16 (d, 1 H, J = 3.6 Hz), 7.36 (d, 1 H, J = 3.6 Hz), 4.54-4.45 (m, 1 H), 2.8-0.7 (m, 52 H). 691.4 (M − 1) |
| 17 | 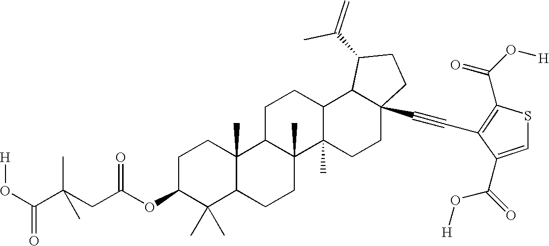 | ¹H NMR (DMSO-d₆, 400 MHz) δ 12.19 (s, 1 H), 7.73 (s, 1 H), 4.73 (s, 1 H, CH═), 4.57 (s, 1 H, CH═), 4.37 (dd, J = 11.2 Hz, J = 4.8 Hz, J = 4.8 Hz, 1 H), 2.70-0.64 (m, 51 H, CH); Mass Spec (m/z): 735.39187 (M + 1), 757.37498 (M + 23). |
| 18 | 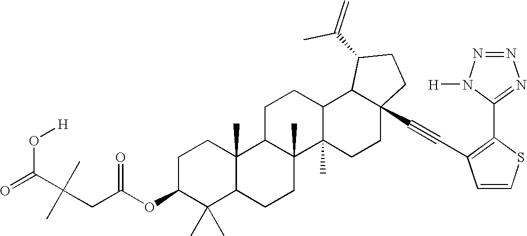 | ¹H NMR (DMSO-d₆, 400 MHz) δ 12.19 (s, 1 H,), 7.87 (s, 1 H, Ar), 7.24 (s, 1 H, Ar), 4.73 (s, 1 H, CH═), 4.58 (s, 1 H, CH═), 4.36 (dd, J = 10.8 Hz, J = 11.2 Hz, J = 4.4 Hz, J = 4.8 Hz, 1 H), 2.70-0.64 (m, 51 H, CH); Mass Spec (m/z): 715.4178 (M + 1), 737.40710 (M + 23). |
| 19 | 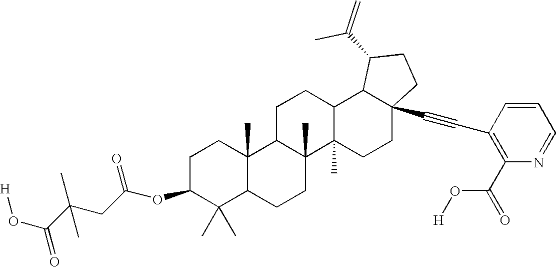 | ¹H NMR (DMSO-d₆, 400 MHz) δ 8.54 (d, J = 4.8 Hz, J = 1.6 Hz, 1 H, Ar), 7.90 (dd, J = 8.0 Hz, J = 1.6 Hz, J = 1.6 Hz, 1 H, Ar), 7.53 (dd, J = 8.4, J = 7.6, Hz, J = 5.2 Hz, J = 4.4 Hz, 1 H, Ar), 4.74 (s, 1 H, CH═), 4.59 (s, 1 H, CH═), 4.37 (dd, J = 11.2 Hz, J = 5.2 Hz, J = 5.2 Hz, 1 H), 2.70-0.64 (m, 51 H, CH); Mass Spec (m/z): 686.4284 (M + 1). |
| 20 | 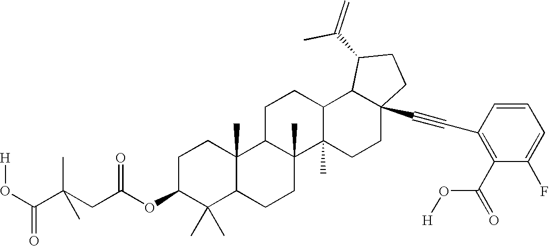 | ¹H NMR (DMSO-d₆, 400 MHz) δ 7.45 (dd, J = 16.0 Hz, J = 7.6 Hz, J = 6.0 Hz, 1 H, Ar), 7.29 (t, J = 18.4 Hz, J = 10.4 Hz, J = 8.0 Hz, 2 H, Ar), 4.75 (s, 1 H, CH═), 4.58 (s, 1 H, CH), 4.37 (dd, J = 11.2 Hz, J = 5.2 Hz, J = 5.2 Hz, 1 H), 2.70-0.64 (m, 52 H, CH); Mass Spec (m/z): 703.4418 (M + 1), 725.4114 (M + 23). |
| 21 | 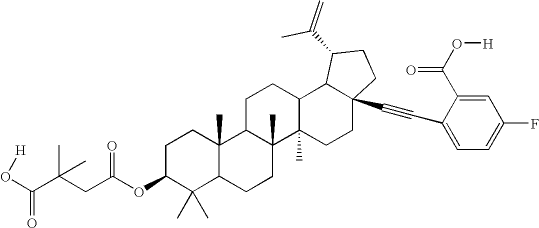 | ¹H NMR (DMSO-d₆, 400 MHz) δ 7.59 (bs, 1 H, Ar), 7.52 (bs, 1 H, Ar), 7.42 (bs, 1 H, Ar), 4.72 (bs 1 H, CH═), 4.58 (bs, 1 H, CH═), 4.35 (bs, 1 H), 2.30-0.64 (m, 51 H, CH); Mass Spec (m/z): 703.4458 (M + 1), 725.4155 (M + 23). |
| 22 | 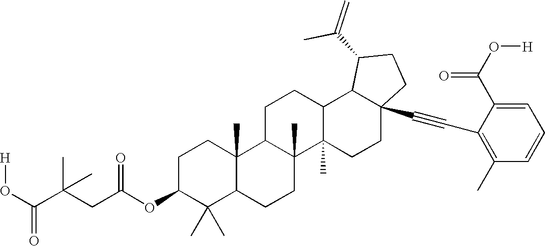 | ¹H NMR (DMSO-d₆, 400 MHz) δ 7.58 (d, J = 8.0 Hz, 1 H, Ar), 7.45 (d, J = 8.0 Hz, 1 H, Ar), 7.28 (t, J = 15.6 Hz, J = 8.0 Hz, J = 7.6 Hz, 1 H, Ar), 4.73 (bs, 1 H, CH═), 4.58 (bs, 1 H, CH═), 4.37 (dd, J = 12.8 Hz, J = 5.2 Hz, J = 5.2 Hz, 1 H), 2.48 (s, 3 H, CH₃), 2.30-0.64 (m, 51 H, CH); Mass Spec (m/z): 699.4675 (M + 1), 721.4371 (M + 23). |
| 23 | 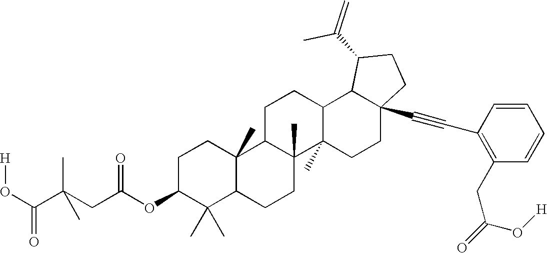 | ¹H NMR (DMSO-d₆, 400 MHz) δ 7.38-7.25 (m, 4 H), 4.75 (s, 1 H), 4.58 (s, 1 H), 4.38 (dd, J = 10.8 Hz, J = 4.4 Hz, J = 4.4 Hz, J = 1 H), 3.75 (s, 2 H), 3.0-0.78 (m, 51 H, CH); Mass Spec (m/z): 706.39032 (M + 1), 729.37953 (M + 23). |
| 24 | 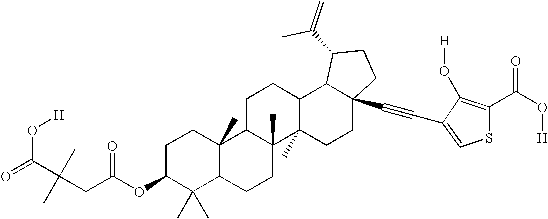 | ¹H NMR (DMSO-d₆, 400 MHz) δ 7.481 (s, 4 H), 6.605 (s, 1 H), 4.769 (s, 1 H), 4.605 (s, 1 H), 4.349 (bs, 1 H), 2.945 (s, 1 H), 3.0- 0.765 (m, 51 H, CH); Mass Spec (m/z): 721.44386 (M + 23). |
| 25 | 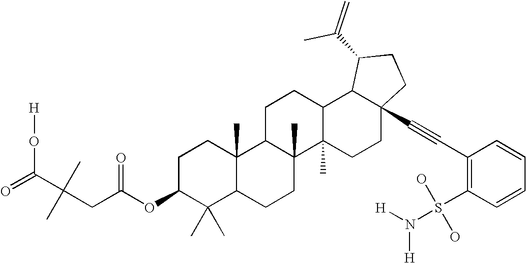 | ¹H NMR (DMSO-d₆, 400 MHz) δ 12.15 (s, 1 H,), 11.86 (s, 1 H), 7.91 (d, J = 8 Hz, 1 H), 7.69- 7.50 (m, 3 H), 4.73 (s, 1 H), 4.73 (s, 1 H), 4.58 (s, 1 H), 3.0-0.64 (m, 53 H, CH); Mass Spec (m/z): 720.4203 (M + 1), 742.4015 (M + 23). |
| 26 | 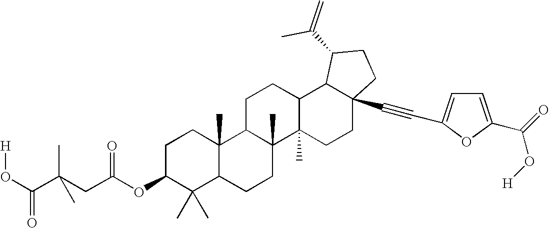 | ¹H NMR (DMSO-d₆, 400 MHz) δ 7.23 (d, J = 3.2 Hz, 1 H,), 6.81 (d, J = 3.6 Hz, 1 H), 4.76 (s, 1 H), 4.60 (s, 1 H), 4.39 (dd, J = 11.2 Hz, J = 4.8 Hz, J = 5.2 Hz, 1 H), 3.0- 0.64 (m, 51 H, CH); Mass Spec (m/z): 674.41825 (M + 1), 697.40748 (M + 23). |
| 27 | 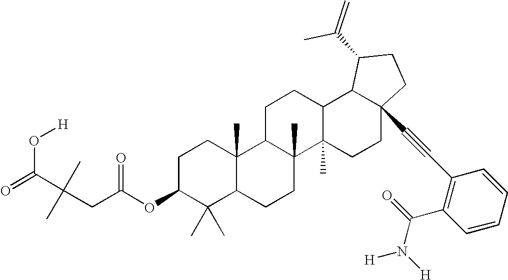 | ¹H NMR (DMSO-d₆, 400 MHz) δ 12.18 (s, 1 H,), 7.68 (s, 1 H) 7.56 (s, 1 H), 7.51 (d, J = 7.6 Hz, 1 H), 7.42 (s, 1 H), 4.74 (s, 1 H), 4.58 (s, 1 H), 4.35 (bs, 3 H), 3.0-0.79 (m, 54 H, CH); Mass Spec (m/z): 684.4513 (M + 1). |
| 28 | 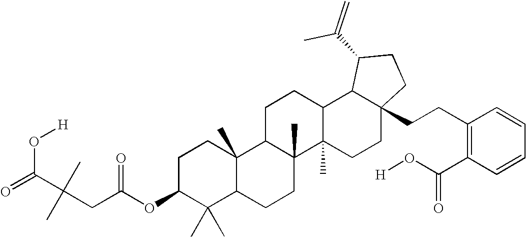 | ¹H NMR (DMSO-d₆, 400 MHz) δ 7.75 (bs, 1 H), 7.46 (bs, 1 H), 7.27 (bs, 2 H), 4.64 (bs, 1 H), 4.51 (bs, 1 H), 4.36 (bs, 1 H), 3.0- 0.78 (m, 57 H, CH); Mass Spec (m/z): 688.47029 (M + 1), 711.45941 (M + 23). |
| 29 | 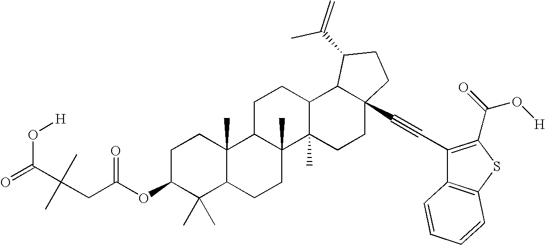 | ¹H NMR (DMSO-d₆, 400 MHz) δ 8.091 (dd, J = 8.8 Hz, J = 3.6 Hz, J = 5.2 Hz, 1 H), 7.955 (bs, 1 H), 7.598 (bs, 2 H), 4.744 (s, 1 H), 4.601 (s, 1 H), 4.371 (bs, 1 H), 3.0- 0.754 (m, 51 H, CH); Mass Spec (m/z): 741.41834 (M + 1), 763.40028 (M + 23). |
| 30 | 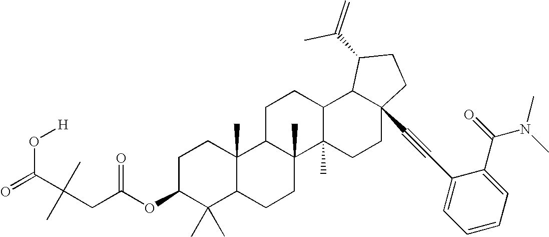 | ¹H NMR (DMSO-d₆, 400 MHz) δ 12.18 (s, 1 H), 7.41-7.38 (m, 3 H), 7.27-7.25 (m, 1 H), 4.74 (s, 1 H), 4.60 (s, 1 H), 4.39 (dd, J = 11.6 Hz, J = 4.8 Hz, J = 4.4 Hz, 1 H), 3.00 (s, 3 H), 2.79 (s, 3 H), 2.54-0.78 (m, 51 H, CH); Mass Spec (m/z): 712.49334 (M + 1), 734.47569 (M + 23). |
| 31 | 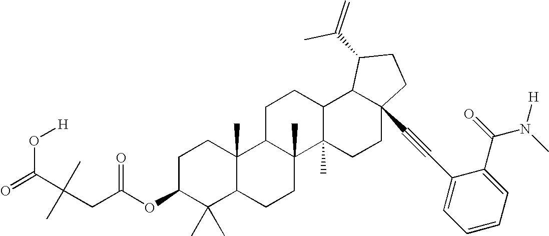 | ¹H NMR (DMSO-d₆, 400 MHz) δ 12.187 (s, 1 H), 8.20 (bt, 1 H), 7.42- 7.37 (m, 4 H), 4.74 (s, 1 H), 4.59 (s, 1 H), 4.39 (dd, J = 11.6 Hz, J = 4.8 Hz, J = 4.8 Hz, 1 H), 2.76 (d, J = 4.4 Hz, 3 H), 2.54-0.78 (m, 52 H, CH); Mass Spec (m/z): 698.47834 (M + 1), 720.46184 (M + 23). |
| 32 | 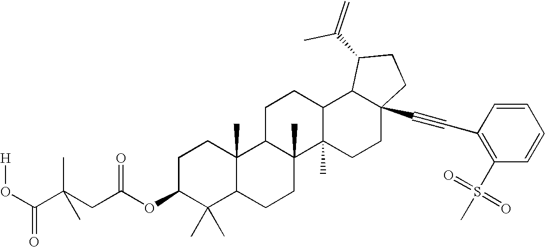 | ¹H NMR (DMSO-d₆, 400 MHz) δ 7.99 (d, J = 9.2 Hz, 1 H), 7.75-7.59 (m, 3 H), 4.74 (s, 1 H), 4.59 (s, 1 H), 4.39 (dd, J = 11.2 Hz, J = 4.8 Hz, J = 5.2 Hz, 1 H), 3.35 (s, 3 H), 3.0-0.78 (m, 54 H, CH); Mass Spec (m/z): 719.43498 (M + 1), 741.41736 (M + 23). |
| 33 | 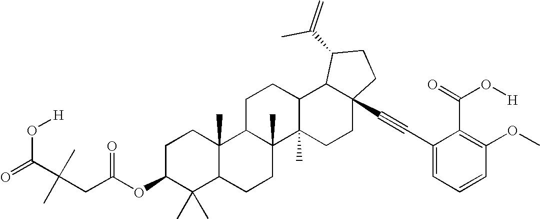 | ¹H NMR (DMSO-d₆, 400 MHz) δ 7.40 (d, J = 8.4 Hz, 1 H), 7.29 (d, J = 3.2 Hz, 1 H), 7.12 (dd, J = 8.0 Hz, J = 2.8 Hz, J = 2.8 Hz, 1 H), 4.73 (s, 1 H), 4.575 (s, 1 H), 4.39 (dd, J = 11.2 Hz, J = 5.2 Hz, J = 5.2 Hz, 1 H), 3.80 (s, 3 H), 3.0-0.78 (m, 51 H, CH); Mass Spec (m/z): 715.45639 (M + 1), 737.43937 (M + 23). |
| 34 | 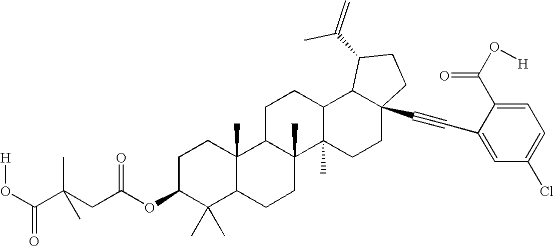 | ¹H NMR (DMSO-d₆, 400 MHz) δ 7.83 (d, J = 8.4 Hz, 1 H), 7.50 (s, 1 H), 7.48 (s, 1 H), 4.74 (s, 1 H), 4.58 (s, 1 H), 4.38 (dd, J = 11.2 Hz, J = 4.8 Hz, J = 5.2 Hz, 1 H), 3.0-0.78 (m, 51 H, CH); Mass Spec (m/z): 719.40704 (M + 1), 741.38892 (M + 23). |
| 35 | 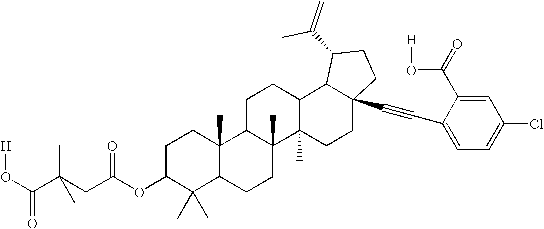 | ¹H NMR (DMSO-d₆, 400 MHz) δ 7.79 (d, J = 2.4 Hz, 1 H), 7.61-7.59 (m, 1 H), 7.49 (d, J = 8.4 Hz, 1 H), 4.73 (s, 1 H), 4.58 (s, 1 H), 4.38 (dd, J = 11.2 Hz, J = 4.8 Hz, J = 5.2 Hz, 1 H), 3.0-0.79 (m, 51 H, CH); Mass Spec (m/z): 719.40667 (M + 1), 741.38873 (M + 23). |
| 36 | 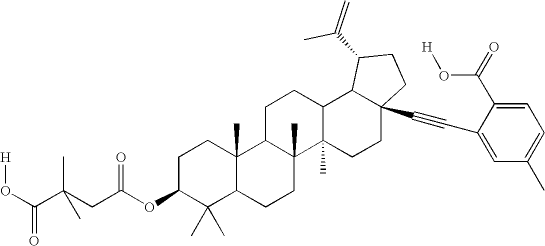 | ¹H NMR (DMSO-d₆, 400 MHz) δ 7.74 (d, J = 8.0 Hz, 1 H), 7.30 (s, 1 H), 7.22 (d, J = 7.2 Hz, 1 H), 4.74 (s, 1 H), 4.58 (s, 1 H), 4.38 (dd, J = 11.2 Hz, J = 4.8 Hz, J = 5.2 Hz, 1 H), 3.0-0.778 (m, 54 H, CH); Mass Spec (m/z): 697.5 (M + 1). |
| 37 | 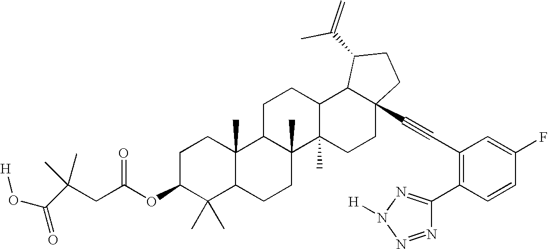 | ¹H NMR (CDCl₃, 400 MHz): 8.81- 8.78 (m, 1 H), 7.67-7.65 (m, 1 H), 7.55-7.46 (m, 1 H), 4.83 (br s, 1 H), 4.70 (br s, 1 H), 4.50-4.56 (m, 1 H), 3.40-3.37 (m, 1 H), 3.08-2.99 (m, 1 H), 2.72-2.52 (m, 2 H), 2.26-0.7 (m, 51 H). Mass Spec (m/z): 727.5 (M + 1). |
| 38 | 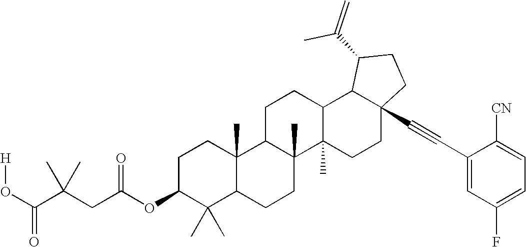 | Mass Spec (m/z): 784.5 (M + 1) |
| 39 | 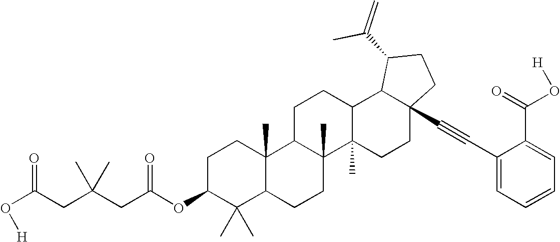 | ¹H NMR (CDCl₃, 400 MHz): 8.065 (d, 1 H, J = 8 Hz), 7.56-7.47 (m, 2 H), 7.38-7.35 (1 H, m), 4.75 (br s, 1 H), 4.61 (br s, 1 H), 4.53-4.56 (m, 1 H), 2.78-2.66 (m, 1 H), 2.55-0.74 (m, 54 H). Mass Spec (m/z): 697.5 (M − 1). |
| 40 | 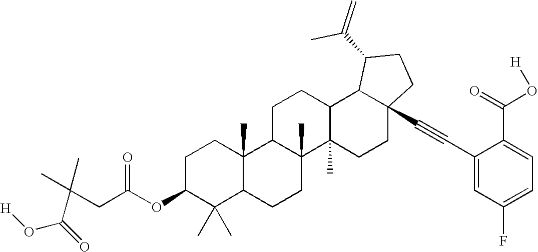 | ¹H NMR (CDCl₃, 400 MHz): 8.09- 8.06 (m, 1 H), 7.21-7.05 (m, 1 H), 7.05-7.0 (m, 1 H), 4.56-4.48 (m, 1 H), 2.78 (d, 1 H, J = 16 Hz), 2.49 (d, 1 H, J = 16 Hz), 2.0-0.58 (m, 55 H). Mass Spec (m/z): 703.5 (M − 1). |
| 41 | 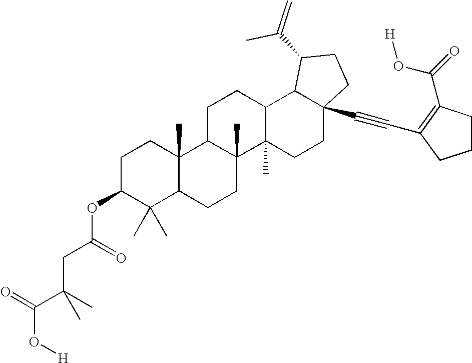 | ¹H NMR (CDCl₃, 400 MHz) δ 4.71 (s, 1 H, CH═), 4.59 (bs, 1 H, CH═), 4.52 (t, J = 8.4 Hz, 1 H), 2.77 (d, J = 15.9 Hz, 1 H), 2.69 (m, 3 H), 2.51 (d, J = 15.9 Hz, 1 H), 2.00-0.600 (m, 52 H, CH); Mass Spec (m/z): 675.46192 (M + 1)⁺, 697.44386 (M + 23)⁺. |
| 42 | 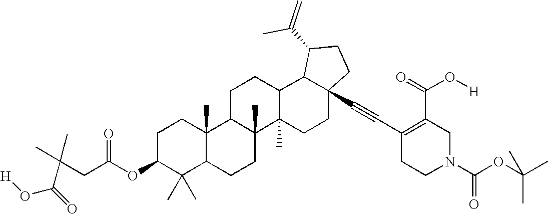 | ¹H NMR (CDCl₃, 400 MHz) δ 4.70 (s, 1 H, CH═), 4.59 (bs, 1 H, CH═), 4.53 (m, 1 H), 4.20 (br, 2 H), 3.5 (br, 2 H), 2.77 (d, J = 15.9 Hz, 1 H), 2.5 (m, 4 H), 2.18 (m, 1 H), 1.88 (m, 4 H), 2.00-0.600 (m, 52 H, CH); Mass Spec (m/z): 789.51797 (M + 1)⁺, 812.50719 (M + 23)⁺. |
| 43 | 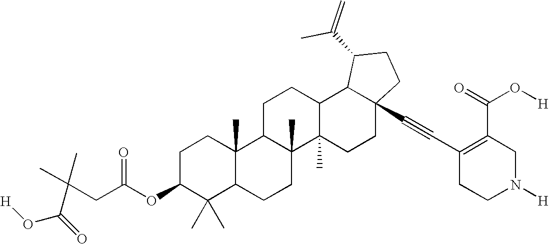 | 1H NMR (DMSO, 400 MHz) δ 13.12 (s, 1 H), 12.19 (s, 1 H), 8.80 (s, 2 H), 4.69 (s, 1 H, ═CH), 4.58 (s, 1 H, ═CH), 4.37 (dd, J1 = 10.8 Hz, J2 = 5.2 Hz, 1 H), 3.84 (s, 2 H), 3.22 (s, 2 H), 2.70-0.70 (m, 52 H, CH); Mass Spec (m/z): 690.4782 (M + 1). 712.4557 (M + 23). |
| 44 | 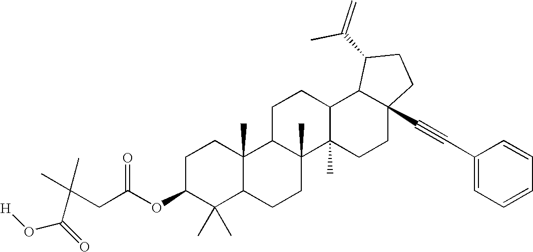 | ¹H NMR (DMSO-d₆, 400 MHz) δ 12.179 (s, 1 H), 7.365 (m, 5 H), 4.740 (s, 1 H), 4.594 (s, 1 H), 4.386 (dd, J = 11.6 Hz, J = 4.8 Hz, J = 4.4 Hz, 1 H), 3.0-0.778 (m, 51 H, CH). |
| 45 | 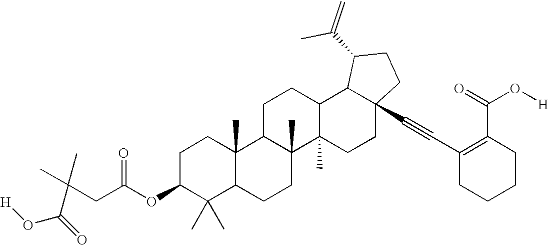 | Mass Spec (m/z): 689.4877 (M + 1). ¹H NMR (DMSO, 400 MHz) δ 12.25 (s, 1 H), 4.70 (d, J = 2.4 Hz, 1 H, ═CH), 4.56 (s, 1 H, ═CH), 4.37 (dd, J₁ = 11.6 Hz, J₂ = 4.8 Hz, 1 H), 2.70-0.70 (m, 60 H, CH); Mass Spec (m/z): 689.47689 (M + H)⁺, 711.45887 (M + Na)⁺. |
| 46 | 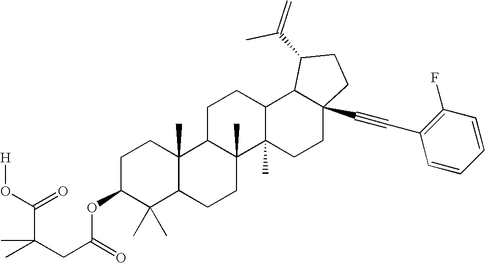 | 1H NMR (DMSO, 400 MHz) δ 12.18 (s, 1 H), 7.45 (dt, J1 = 7.6 Hz, J2 = 1.6 Hz, 1 H), 7.40 (m, 1 H), 7.27 (dt, J1 = 10 Hz, J2 = 1.2 Hz, 1 H), 7.20 (dt, J1 = 7.6 Hz, J2 = 1.2 Hz, 1 H), 4.73 (d, J = 2.4 Hz, 1 H, ═CH), 4.59 (s, 1 H, ═CH), 4.37 (dd, J1 = 11.2 Hz, J2 = 4.8 Hz, 1 H), 2.70-0.70 (m, 51 H, CH); Mass Spec (m/z): 681.42923 (M + Na)+. |
| 47 | 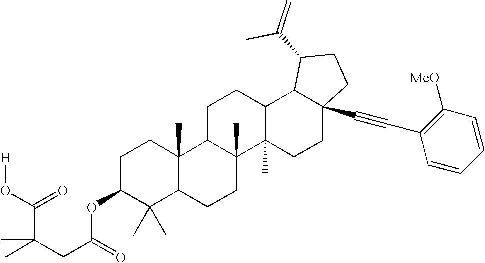 | 1H NMR (DMSO, 400 MHz) δ 12.18 (s, 1 H), 7.30 (m, 1 H), 7.40 (m, 1 H), 7.01 (dt, J1 = 8.4 Hz, J2 = 0.8 Hz, 1 H), 6.90 (dt, J1 = 7.6 Hz, J2 = 1.2 Hz, 1 H), 4.72 (d, J = 2.4 Hz, 1 H, ═CH), 4.58 (s, 1 H, ═CH), 4.37 (dd, J1 = 11.6 Hz, J2 = 5.2 Hz, 1 H), 3.80 (s, 3 H), 2.70-0.70 (m, 51 H, CH); Mass Spec (m/z): 693.44818 (M + Na)+. |
| 48 | 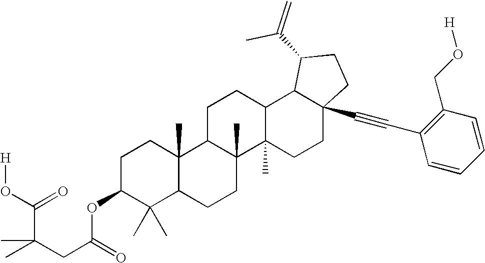 | 1H NMR (DMSO, 400 MHz) δ 7.50 (d, J = 7.6 Hz, 1 H), 7.34 (m, 2 H), 7.23 (t, J = 7.6 Hz, 1 H), 4.74 (d, J = 2.0 Hz, 1 H, ═CH), 4.67 (s, 2 H), 4.59 (s, 1 H, ═CH), 4.37 (dd, J1 = 11.2 Hz, J 2 = 4.8 Hz, 1 H), 2.70-0.70 (m, 51 H, CH); Mass Spec (m/z): 671.46664 (M + H)+, 693.44936 (M + Na)+. |
| 49 | 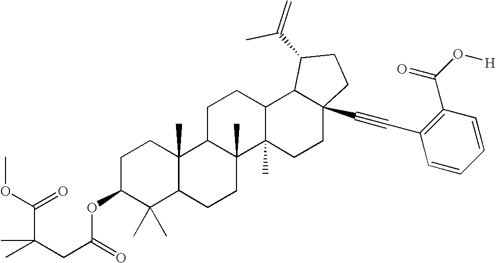 | 1H NMR (CDCl₃, 400 MHz) δ 4.73 (s, 1 H, ═CH), 4.60 (s, 1 H, ═CH), 4.48 (dd, J1 = 10.8 Hz, J2 = 5.6 Hz, 1 H), 3.69 (s, 3 H), 2.70-0.70 (m, 60 H, CH); Mass Spec (m/z): 703.49277 (M + H)+, 725.47474 (M + Na)+. |
| 50 | 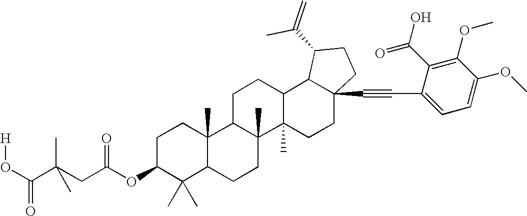 | 1H NMR (DMSO-d₆, 400 MHz) δ 7.33 (s, 1 H), 6.91 (s, 1 H), 4.71 (bs, 1 H), 4.58 (bs, 1 H), 4.386 (dd, J = 11.6 Hz, J = 4.8 Hz, J = 4.4 Hz, 1 H), 3.81 (s, 3 H), 3.80 (s, 3 H), 3.0-0.778 (m, 55 H, CH); Mass Spec (m/z): 745.4549 (M + 1). |
| 51 | 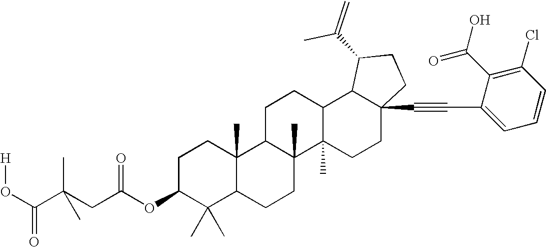 | 1H NMR (DMSO-d₆, 400 MHz) δ 13.686 (s, 1 H), 12.184 (s, 1 H), 7.508-7.402 (m, 3 H), 4.745 (bs, 1 H), 4.586 (bs, 1 H), 4.385 (dd, J = 11.6 Hz, J = 4.8 Hz, J = 4.8 Hz, 1 H), 3.0-0.778 (m, 51 H, CH); Mass Spec (m/z): 719.3978 (M + 1), 741.39010 (M + 23). |
| 52 | 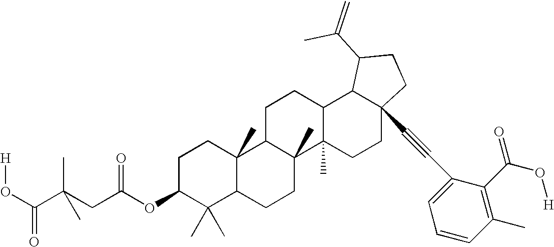 | 1H NMR (DMSO-d₆, 400 MHz) δ 7.309-7.216 (m, 3 H), 4.738 (s, 1 H), 4.581 (s, 1 H), 4.386 (dd, J = 11.2 Hz, J = 5.2 Hz, J = 5.6 Hz, 1 H), 3.0-0.778 (m, 58 H, CH); Mass Spec (m/z): 699.46295 (M + 1), 721.44612 (M + 23) |
| 53 | 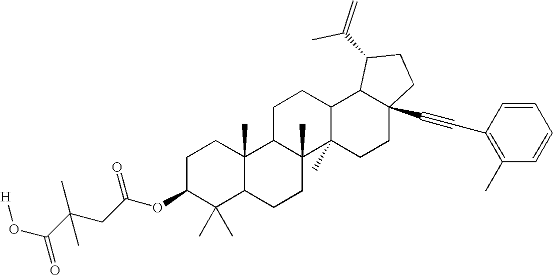 | 1H NMR (DMSO-d₆, 400 MHz) δ 12.219 (s, 1 H), 7.341-7.165 (m, 4 H), 4.730 (s, 1 H), 4.590 (s, 1 H), 4.386 (dd, J = 11.2 Hz, J = 4.8 Hz, J = 5.2 Hz, 1 H), 3.346 (s, 3 H), 3.0-0.778 (m, 51 H, CH); |
| 54 | 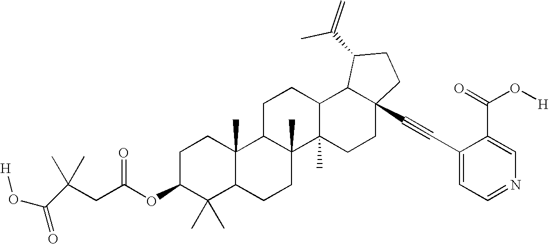 | ¹H NMR (DMSO-d₆, 400 MHz) 8.71 (br s, 1 H), 8.62 (br d, 1 H, J = 4.4 Hz), 7.70 (br d, 1 H, J = 4.4 Hz), 4.74 (br s, 1 H), 4.86 (br s, 1 H), 4.36 (br d, 1 H, J = 7.2 Hz), 2.8-0.7 (51 H). Mass Spec (m/z): 686.4488 (M + 1). |
| 55 | 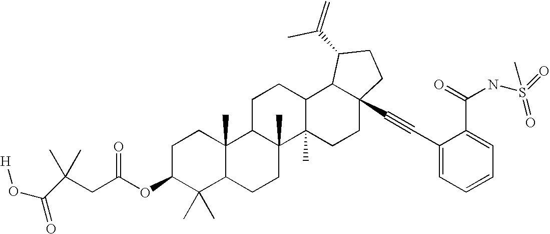 | 1H NMR (DMSO, 400 MHz) δ 12.12 (s, 1 H), 7.56-7.42 (m, 2 H), 4.74 (s, 1 H), 4.58 (s, 1 H), 4.37 (m, 1 H), 3.35 (s, 3 H), 2.70-0.70 (m, 54 H, CH); Mass Spec (m/z): 762.43980 (M + H)+, 784.42175 (M + Na)+. |

### Example 2

### Determination of Antiviral Activity

The compounds of the invention can be tested in the following MT-4 assay to detect antiviral activity.

### 1. MT-4 Cytoprotection Assay

The HTLV-1 transformed T cell line, MT-4, is highly susceptible to HIV-1 infection. Anti-HIV-1 agents were evaluated in this target cell line by protection from the HIV-induced cytopathic effect. In this assay, viability of both HIV-1 and mock-infected cells was assessed in a colorimetric assay that monitors the ability of metabolically-active cells to reduce the tetrazolium salt WST-1. Cytoprotection by antiviral compounds is indicated by the positive readout of increased WST-1 cleavage.

Briefly, exponentially growing MT-4 cells were mock-infected or batch-infected with the HIV-1 laboratory strain, NL4-3, at a multiplicity of infection of 0.0005. Following a two hour infection, the cells were washed to remove unbound virus and plated in the presence of increasing concentrations of compound. After four days incubation, cytoprotection in the infected cells and compound toxicity in mock-infected cells were analyzed using the WST-1 assay.

It was found that all compounds in Table 2 showed antiviral activity in the MT4 assay with an EC₅₀ of about 50 μM or less in the assay. Compounds 6, 9, 11, 13, 15, 16, 18, 20-23, 27, 28, 30, 32-36, 40-42, 43, 45, 50, 51, 54 and 55 are most active and all have an EC₅₀ of 100 nM or less in the assay.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound according to Formula I 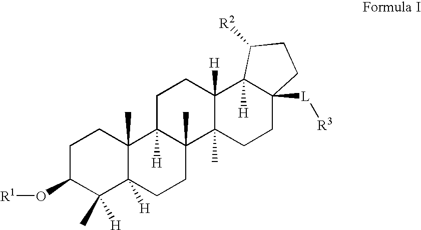
and pharmaceutically acceptable salts thereof,
wherein:
L is ethylene or ethynylene;
R¹ is hydro, R¹¹—C(O)—, —S(O)R¹¹ or —S(O)OR¹¹, wherein R¹¹ is C₁₋₂₀ alkyl, C₂₋₂₀ alkenyl, or C₂₋₂₀ alkynyl each being optionally substituted with one or more substituents independently chosen from the group of:
(1) —C(O)R¹² where R¹² is —OH, C₁₋₆ alkoxy, C₁₋₆ alkenyloxy, C₁₋₆ alkynyloxy, and C₃₋₆ cycloalkoxy;
(2) carboxyalkoxy;
(3) —C(O)—N(R¹³)(R¹⁴) where R¹³ and R¹⁴ are independently hydro, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is hydro, C₁₋₆ alkyl or aryl, or R¹³ and R¹⁴ together with the nitrogen atom they are linked to form a 3 to 6-membered heterocycle;
(4) —N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
(5) —SO₃R¹⁵ where R¹⁵ is C₁₋₆ alkyl, aryl or heteroaryl;
(6) —NHSO₃R¹⁶ where R¹⁶ is C₁₋₆ alkyl, aryl, or heteroaryl; and
(7) —P(O)(OR¹⁷)₂ where R¹⁷ is H or C₁₋₆ alkyl,
wherein optionally two substituents at one carbon atom of R¹¹ together with the one carbon atom they are attached to, form a 3 to 6-membered cycloalkyl;
R² is isopropenyl or isopropyl, optionally substituted with one or two substituents independently selected from hydroxyl, halo, amino, pyrrolidinyl, and piperidinyl; and
R³ is hydro or an optionally substituted aryl, heteroaryl, carbocycle or heterocycle.

2. The compound according to claim 1, wherein when L is ethylene, R³ is an optionally substituted aryl.

3. The compound according to claim 1, wherein L is ethynylene.

4. The compound according to claim 1, wherein R¹ is C₄₋₈ carboxyalkanoyl, C₄₋₈ carboxyalkenoyl, or C₄₋₈ carboxyalkoxyalkanoyl.

5. The compound according to claim 1, wherein R¹ is chosen from the group consisting of:

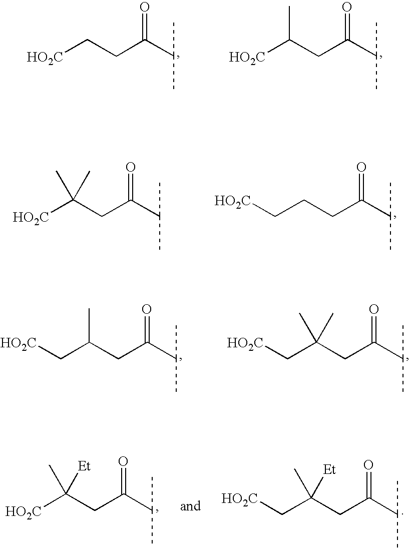

6. The compound according to claim 1, wherein R¹ is —C(═O)—(CH₂)ₘ—C(CH₃)₂—COOH or —C(═O)—(CH₂)ₘ—C(CH₃)₂—(CH₂)ₙ COOH, wherein m and n are independently an integer from 0-10.

7. The compound according to claim 1, wherein R¹ is 3′,3′-dimethylsuccinyl or 3′,3′-dimethylglutaryl.

8. The compound according to claim 1, wherein R² is isopropenyl, isopropyl, 1′-hydroxyisopropyl, 2′-hydroxyisopryl, 1′,2′-dihydroxyisopropyl, or 1′-pyrrolidinyl-2′-hydroxyisopropyl.

9. The compound according to claim 8, wherein R² is —C(═CH₂)—CH₃ or —CH(CH₃)₂.

10. The compound according to claim 1, wherein R³ is an aryl, heteroaryl, carbocycle or heterocycle having at least one substituent chosen from the group consisting of hydroxyl, halo, C₁₋₆ alkyl, C₁₋₆ alkoxy, C₁₋₆ alkylthio, carboxylic acid, C-carboxy, C-amido, aminosulfonyl, sulfonyl, and —COOH bioisosteres.

11. The compound according to claim 10, wherein R³ is phenyl, thiophenyl, furanyl, benzofuranyl, benzothiophenyl, isoxazolyl, pyridinyl, pyrazinyl, pyrimidinyl, 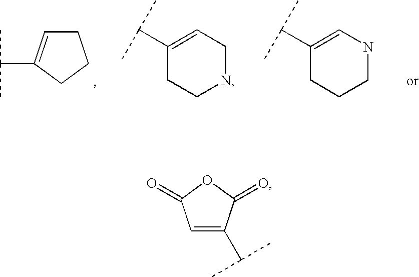
optionally having a substituent chosen from the group consisting of hydroxyl, halo (F, Cl, Br, I), C₁₋₆ alkyl, C₁₋₆ alkoxy, C₁₋₆ alkylthio, carboxylic acid, C-carboxy, C-amido, aminosulfonyl, sulfonyl, and —COOH bioisosteres.

12. The compound according to claim 1, wherein R³ is an aryl, heteroaryl, carbocycle or heterocycle having a substitutent at an ortho position relative to the position where L (ethynylene or ethylene) is attached to, chosen from hydroxyl, mercapto, C₁₋₆ alkoxy, C₁₋₆ haloalkoxy, C₁₋₆ alkylthio, carboxylic acid, carboxylic acid bioisosteres, carboxyalkyl, carboxyalkenyl, carboxyalkynyl, carboxyalkoxy, alkanoyl, alkylthiocarnonyl, carboxylalkoxyalkanoyl, C-carboxy, O-carboxy, O-carbamyl, N-carbamyl, O-thiocarbamyl, N-thiocarbamyl, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH).

13. The compound according to claim 1, wherein R³ is an aryl, heteroaryl, carbocycle or heterocycle substituted with
(1) a first substitutent at an ortho position relative to the position where L (ethynylene or ethylene) is attached to, wherein the first substituent is chosen from hydroxyl, mercapto, C₁₋₆ alkoxy, C₁₋₆ haloalkoxy, C₁₋₆ alkylthio, carboxylic acid, carboxylic acid bioisosteres, carboxyalkyl, carboxyalkenyl, carboxyalkynyl, carboxyalkoxy, alkanoyl, alkylthiocarnonyl, carboxylalkoxyalkanoyl, C-carboxy, O-carboxy, O-carbamyl, N-carbamyl, O-thiocarbamyl, N-thiocarbamyl, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH), and optionally
(2) one or more (1, 2, 3 or 4) additional substituents independently chosen from the group of hydroxyl, mercapto, halo (F, Cl, Br, or I), cyano, nitro, C₁₋₆ alkyl, C₂₋₆ alkenyl, C₂₋₆ alkynyl, C₁₋₆ haloalkyl, C₁₋₆ alkoxy, C₁₋₆ haloalkoxy, C₁₋₆ alkylthio, C₁₋₆ hydroxyalkyl, C₁₋₆ thioalkyl, alkoxyalkyl, carboxylic acid, carboxylic acid bioisosteres, carboxyalkyl, carboxyalkoxy, carboxyalkenyl, carboxyalkynyl, alkanoyl, alkylthiocarnonyl, carboxylalkoxyalkanoyl, C-carboxy, O-carboxy, O-carbamyl, N-carbamyl, O-thiocarbamyl, N-thiocarbamyl, amino, aminoalkyl, alkylamino, C-amido, N-amido, sulfonic acid, sulfonamidecarbonyl, alkanoylaminosulfonyl, aminosulfonyl, sulfonyl, sulfonamide, hydroxyaminocarbonyl, alkoxyaminocarbonyl, aminothiocarbonyl, aminosulfonyloxy, and —O—S(═O)₂(OH).

14. The compound according to claim 1, having an IC₅₀ of less than about 500 nM as determined in the MT4 assay in Example 2.

15. The compound according to claim 1, chosen from

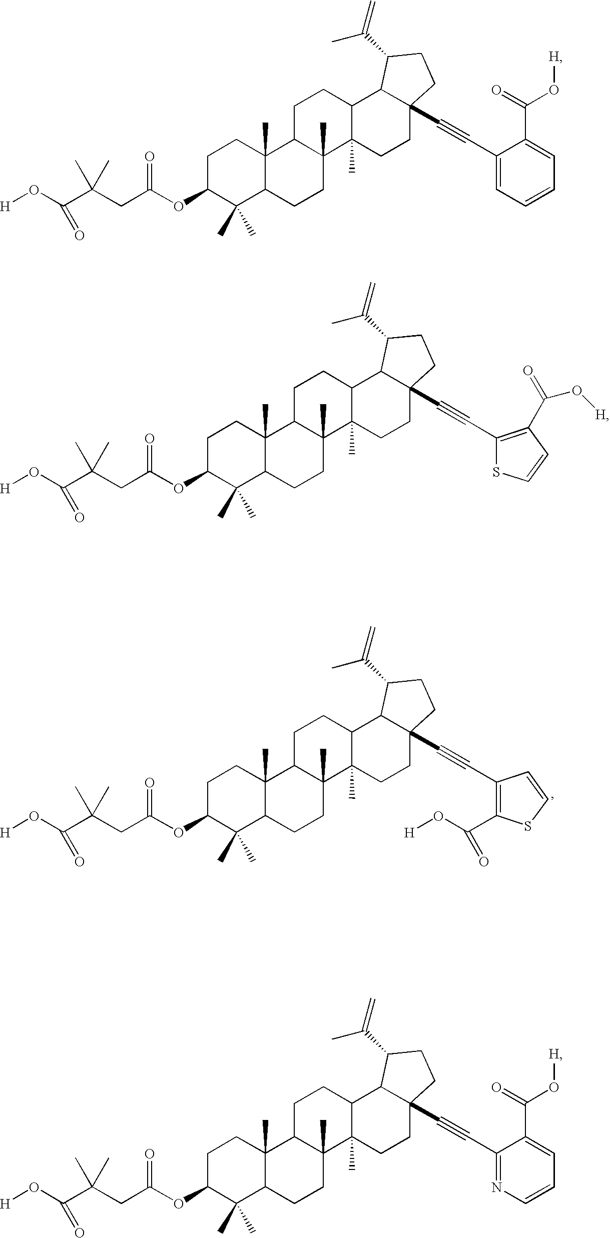

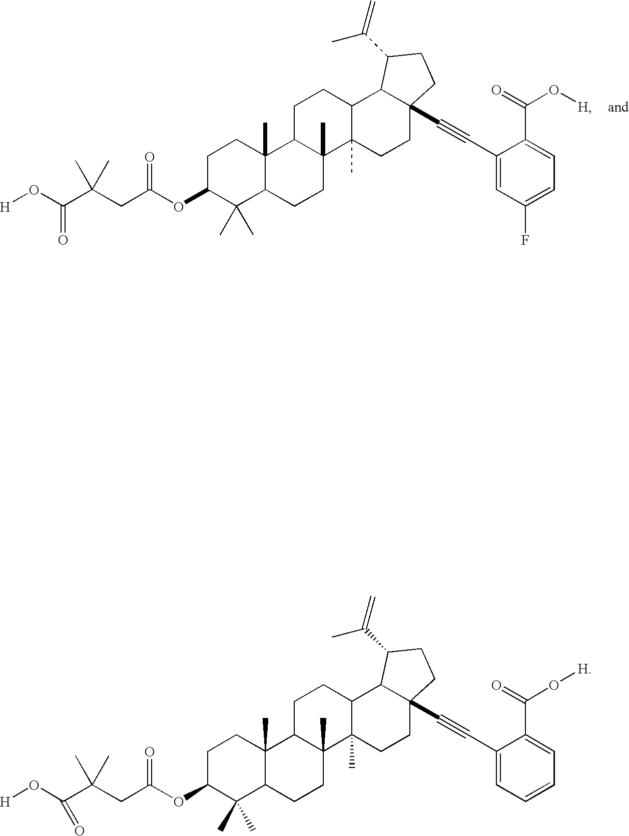

16. A pharmaceutical composition comprising a therapeutically effective amount of a compound according to claim 1, and a pharmaceutically acceptable carrier.

17. A method of inhibiting HIV maturation in a cell infected with HIV, said method comprising:
contacting the cell with a compound according to claim 1.

18. The compound according to claim 1 having a structure according Formula IIa 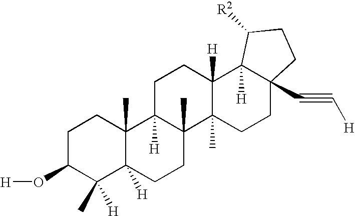
wherein R² is defined as in claim 1.

19. A method of making a compound, comprising:
providing a compound according to Formula II 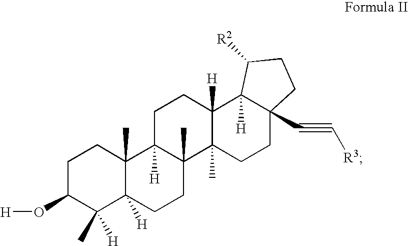
and
reacting the compound of Formula II with a reagent under conditions sufficient to convert the hydroxyl group at C-3 position to a moiety of R¹—O—, wherein R¹ is hydro, R¹¹—C(O)—, —S(O)R¹¹ or —S(O)OR¹¹, wherein R¹¹ is C₁₋₂₀ alkyl, C₂₋₂₀ alkenyl, or C₂₋₂₀ alkynyl each being optionally substituted with one or more substituents independently chosen from the group of:
(1) —C(O)R¹² where R¹² is —OH, C₁₋₆ alkoxy, C₁₋₆ alkenyloxy, C₁₋₆ alkynyloxy, and C₃₋₆ cycloalkoxy;
(2) carboxyalkoxy;
(3) —C(O)—N(R¹³)(R¹⁴) where R¹³ and R¹⁴ are independently hydro, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is hydro, C₁₋₆ alkyl or aryl, or R¹³ and R¹⁴ together with the nitrogen atom they are linked to form a 3 to 6-membered heterocycle;
(4) —N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
(5) —SO₃R¹⁵ where R¹⁵ is C₁₋₆ alkyl, aryl or heteroaryl;
(6) —NHSO₃R¹⁶ where R¹⁶ is C₁₋₆ alkyl, aryl, or heteroaryl; and
(7) —P(O)(OR¹⁷)₂ where R¹⁷ is H or C₁₋₆ alkyl,
wherein optionally two substituents at one carbon atom of R¹¹ together with the one carbon atom they are attached to, form a 3 to 6-membered cycloalkyl.

20. The compound of claim 1 having the formula [chemical structure]
